(19) United States
(12) Patent Application Publication
(10) Pub. No.: US 2005/0181037 A1
(43) Pub. Date: Aug. 18, 2005

(54) Cardiolipin compositions their methods of preparation and use

(75) Inventors: Moghis U. Ahmad, Wadsworth, IL (US); Zhen Lin, Gurnee, IL (US); Shoukath M. Ali, Lake Bluff, IL (US); Imran Ahmad, Wadsworth, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: NEOPHARM, INC., Lake Forest, IL (US)

(21) Appl. No.: 10/996,536

(22) Filed: Nov. 23, 2004

Related U.S. Application Data
(63) Continuation of application No. PCT/US03/16412, filed on May 23, 2003.
(63) Continuation-in-part of application No. PCT/US03/13917, filed on May 4, 2003.
(60) Provisional application No. 60/438,659, filed on Jan. 7, 2003.
(60) Provisional application No. 60/429,285, filed on Nov. 26, 2002.
(60) Provisional application No. 60/419,277, filed on Oct. 16, 2002.
(60) Provisional application No. 60/383,340, filed on May 24, 2002.
(60) Provisional application No. 60/438,659, filed on Jan. 7, 2003.

(51) Int. Cl. A61K 48/00; A61K 38/16; C07H 21/04; C07K 14/47; A61K 9/127; A61K 31/675

(52) U.S. Cl. 424/450; 514/44; 514/7; 514/25; 514/79; 514/124; 530/324; 536/23.1; 554/79; 435/458

(57) ABSTRACT

The invention provides new synthetic routes for cardiolipin with different fatty acids and/or alkyl chains with varying chain length and also with or without unsaturation. The reaction schemes can be used to generate new forms of cardiolipin, including cardiolipin variants. The cardiolipin prepared by the present methods can conveniently be incorporated into liposomes and other lipid formulations that can also include active agents such as hydrophobic or hydrophilic drugs. Such formulations can be used to treat diseases or in diagnostic and/or analytical assays. Liposomes also can include ligands, e.g., for targeting them to a cell type or specific tissue.

### FIELD OF THE INVENTION

This invention pertains to novel cardiolipin compositions, to methods for preparing them and to liposome compositions that contain them. The invention also pertains to liposome formulations or complexes or emulsions containing active agents and their use in diagnostic assays and in the treatment of diseases in humans and animals.

### BACKGROUND OF THE INVENTION

Liposomal formulations have the capacity to increase the solubility of hydrophobic drugs in aqueous solution. They often reduce the side effects associated with drug therapy and they provide flexible tools for developing new formulations of active agents.

Liposomes are commonly prepared from natural phospholipids such as phosphatidylcholine, phosphatidylethanolamine, phosphatidylserine, phosphatidylglycerol, phosphatidic acid, and phosphatidylinositol. Anionic phospholipids, such as phosphatidyl glycerol and cardiolipin, can be added to generate a net negative surface charge that provides for colloid stabilization. These components are often purified from natural sources and in some cases they can be chemically synthesized.

Cardiolipin (also known as diphosphatidyl glycerol), constitutes a class of complex anionic phospholipids that is typically purified from cell membranes of tissues associated with high metabolic activity, including the mitochondria of heart and skeletal muscles. However, known chromatographic purification techniques cannot resolve cardiolipin into discrete molecular species. Therefore, drug formulations containing this component are not homogeneous.

Homogeneous tetramyristoylcardiolipin can be obtained through chemical synthesis. However, the availability of this compound is limited and it is currently too expensive for general use in drug formulations. Other homogeneous cardiolipin species having defined hydrophobic acyl groups, such as fatty acids, are either not available commercially or are available only in small quantities and at substantial cost.

The limited availability of cardiolipin is due, in part, to the fact that methods for synthesizing it are cumbersome, time consuming, and expensive. Generally, they involve the stepwise buildup of individual parts of the molecule starting from various derivatives of substituted glycerol and multiple intermediate purifications. Known synthetic methodologies are mainly divided in two groups: (a) coupling the primary hydroxyl groups of a 2-O-protected glycerol with 1,2-O-diacyl-sn-glycerol using a phosphorylating agent and (b) condensation at both primary hydroxyl groups of a 2-O-protected glycerol and phosphatidic acid in the presence of 2,4,6-triisopropylbenzenesulfonyl chloride (TPS) and pyridine.

Ramirez et al. (Synthesis, 769-770 (1976); Tetrahedron, 33: 599-608 (1977)) describe a synthetic method for cardiolipin in which a 1,2-O-diacylglycerol is phosphorylated with di(1,2-dimethylethenylene) pyrophosphate in the presence of triethylamine. The product is reacted with 2-tert-butyldimethylsilyl chloride glycerol in the presence of an amine catalyst to produce the phosphotriester form of cardiolipin. The acetonyl phosphate protecting group is removed under mild basic conditions to give the phosphodiester and the silyl group is removed under mild acid conditions. This synthetic method suffers from a significant transesterification reaction that generates a number of side products in addition to the desired cardiolipin.

Duralski et al. (Tetrahedron Lett., 39: 1607-1610 (1998)) describe the protection of the primary hydroxyl groups of glycerol by reaction with 4,4′-dimethoxytritylchloride. The 2-hydroxyl group was then protected by reaction with tert-butyldimethylsilyl chloride and the trityl groups were removed by treatment with p-toluenesulphonic acid. The primary hydroxyl group of a diacylglycerol was phosphorylated using the bifunctional phosphorylating agent 2-chlorophenyl phosphorodi-(1,2,4-triazolide) which was generated in situ from a mixture containing 1,2,4-triazole and 2-chlorophenyl phosphodichloridate in the presence of triethylamine and allowed to react with the silylated glycerol in the presence of 2,4,6-triisopropylsulphonyl chloride and N-methylimidazole. The fully protected cardiolipin was deprotected by treatment with 2-nitrobenzaldoxirrie and N,N,N,N-tetramethylguanidine and the silyl groups removed by treatment with acetic acid.

Saunders and Schwarz (J. Am. Chem. Soc., 88: 3844-3847 (1966)) described the preparation of cardiolipin by phosphorylating 2,3-di-O-stearoyl-D-glycerol with phosphorous oxychloride, adding 2-O-benzylglycerol and removing the benzyl group by catalytic hydrogenation. The product was purified by silicic acid chromatography. This method was later questioned by investigators (Ramirez et al., Tetrahedron, 33: 599-608 (1977)) who were unable to use it to successfully obtain cardiolipin.

Mishina et al. (Bioorg. Khim., 11: 992-994 (1985); Bioorg. Khim., 13: 1110-1115 (1987)) utilized oleic imidazole to acylate sn-glycero-3-phosphocholine which was then cleaved with cabbage phospholipase D to give 1,2-dioleoyl-sn-glycero-3-phosphoric acid. This compound was condensed with 2-O-tert-butyldimethylsilyl glycerol in pyridine containing 2,4,6-triisopropylbenzenesulphonyl chloride to yield the protected cardiolipin which was deprotected by standard methods to produce cardiolipin.

Stepanov et al. (Zh. Org. Khim, 20: 985-988 (1984)) describe the condensation of diacyl phosphatidic acid with 2-O-benzyl glycerol using the condensing agent, 2,4,6-triisopropylbenzenesulphonyl chloride.

Keana et al. (J. Org. Chem., 51: 2297-2299 (1986)) describe the coupling of a phosphatidylglycerol (PG) methyl ester with a phosphatidic acid (PA) in pyridine using 2,4,6-triisopropylbenzenesulphonyl chloride. The monomethyl ester of coupling product was methylated with diazomethane to yield dimethyl ester of cardiolipin which underwent demethylation with NaI to give cardiolipin.

Cardiolipin has also been generated via a reaction between the silver salt of diacylglycerophosphoric acid benzyl ester with 1,3-diiodopropanol benzyl ether or 1,3-diiodopropanol t-butyl ether. For example, De Haas and van Deenen (Biochim. Biophys. Acta, 116: 114-124 (1966)) used a multi-step sequence to obtain cardiolipin in an overall yield of 26%. Reaction between silver benzyldiacyl-L-α-glycerophosphate and 2-tert-butoxy-1,3-diiodoglycerol in a 2:1 molar ratio yielded a triester which was purified and deprotected to yield cardiolipin. Benzyl protecting groups were removed from the triester intermediate by treatment with barium iodide and the tert-butyl-ether group was removed by treatment with anhydrous HCl in chloroform. Similarly, Inoue et al. (Chem. Pharm. Bull., 11: 1150-1156 (1963)) described a method for preparing a 1,3-propanediol analogue of cardiolipin in which the phosphodiester linkages were prepared by the reaction of 1,3-diiodopropane with silver benzyldiacyl-L-α-glycero-phosphate and removal of the benzyl protecting group with sodium iodide. Although these later schemes were suitable for the preparation of small quantities of cardiolipin, it is unattractive for the routine preparation of large quantities due to the many steps involved, the requirement for careful purification of intermediates and the use of highly photosensitive silver salt intermediates and unstable iodo intermediates.

New synthetic methods are needed that can be used to prepare large quantities of diverse and homogeneous cardiolipin species in a more cost effective manner. Such methods would increase the availability of a wider variety of homogeneous cardiolipin species and would diversify the lipids available for development of new liposomal formulations of active agents which will have more defined compositions than is presently possible.

The invention provides such methods and compositions. These and other advantages of the invention, as well as additional inventive features, will be apparent from the description of the invention provided herein.

### BRIEF SUMMARY OF THE INVENTION

The invention provides novel cardiolipin molecules and analogues and new synthetic routes for cardiolipin with different fatty acids and/or alkyl chains with varying chain length and saturation/unsaturation. The reaction schemes can be used to generate new forms of cardiolipin, including cardiolipin variants. The cardiolipin prepared by the present methods can conveniently be incorporated into liposomes, emulsions or complexes (e.g., drug complexes) that can also include (e.g., complexed with or entrapped within liposomes) active agents such as genes and gene vectors, antisense molecules (e.g., oligonucleotides), proteins and peptides, protein or chemical drugs (e.g., hydrophobic or hydrophilic drugs) or diagnostic agents. Such liposomes and other formulations can be used to treat diseases or in diagnostic and/or analytical assays.

### BRIEF DESCRIPTION OF THE DRAWINGS

FIG. 1 shows the general structure for cardiolipin.

FIG. 2 shows one synthetic scheme for cardiolipin.

FIG. 3 shows an alternative synthetic scheme for cardiolipin.

FIG. 4 shows an alternative synthetic scheme for cardiolipin ether analogue.

FIG. 5 shows an alternative synthetic scheme for cardiolipin.

FIG. 6 shows an alternative synthetic scheme for cardiolipin.

FIG. 7 shows an alternative synthetic scheme for cardiolipin.

FIG. 8 shows an alternative synthetic scheme for cardiolipin.

### DETAILED DESCRIPTION OF THE INVENTION

The present invention provides a cardiolipin molecules (including analogues or derivative thereof). In one embodiment, the cardiolipin molecule has a structure according to the following general formula: 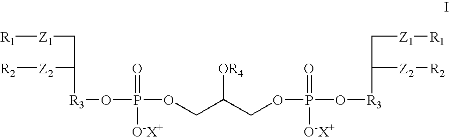
wherein Z₁ and Z₂ are the same or different and are —O—C(O)—, —O—, —S—, —NH—C(O)— or the like;
R₁ and R₂ are the same or different and can be either H, or a saturated or unsaturated alkyl group;
R₃ is (CH₂)ₙ and n=0-10;
R₄ is hydrogen, alkyl, substituted alkyl, cycloalkyl, substituted cycloalkyl, a peptide, dipeptide, polypeptide, protein, carbohydrate such as glucose, mannose, galactose, polysaccharide and the like, heterocyclic, nucleoside, polynucleotide and the like;
X is a cation, and most preferably a non-toxic cation such as hydrogen, ammonium, sodium, potassium, calcium, barium ion and the like.

In preferred embodiments, the cardiolipin analogue can have formula II or formula III:

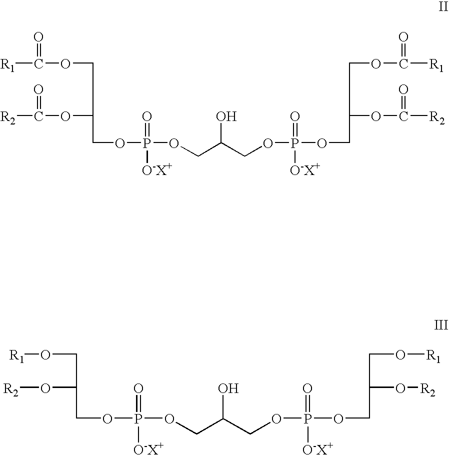

The present invention also provides compositions comprising a cardiolipin analogue having a structure according to the following general formula: 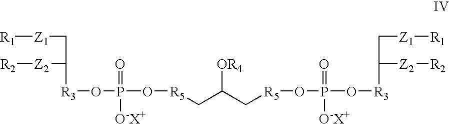
wherein Z₁ and Z₂ are the same or different and are —O—C(O)—, —O—, —S—, —NH—C(O)— or the like;
R₁ and R₂ are the same or different and are H, or saturated or unsaturated alkyl group;
R₃ is (CH₂)ₙ and n=0-10;
R₄ is hydrogen, alkyl, substituted alkyl, cycloalkyl, substituted cycloalkyl, a peptide, dipeptide, polypeptide, protein, carbohydrate such as glucose, mannose, galactose, polysaccharide and the like, heterocyclic, nucleoside, polynucleotide and the like;
R₅ is a linker, which can comprise alkyl, substituted alkyl, cycloalkyl, substituted cycloalkyl, alkyloxy, polyalkyloxy such as pegylated ether of containing from about 1 to about 500 alkyloxy mers (and can have at least about 10 alkyloxy mers, such as at least about 50 alkyloxy mers or at least about 100 alkyloxy mers, such as at least about 200 alkyloxy mers or at least about 300 alkyloxy mers or at least about 400 alkyloxy mers), substituted polyalkyloxy and the like, a peptide, dipeptide, polypeptide, protein, carbohydrate such as glucose, mannose, galactose, polysaccharides and the like;
X is a cation, and most preferably a non-toxic cation such as hydrogen, ammonium, sodium, potassium, calcium, barium ion and the like.

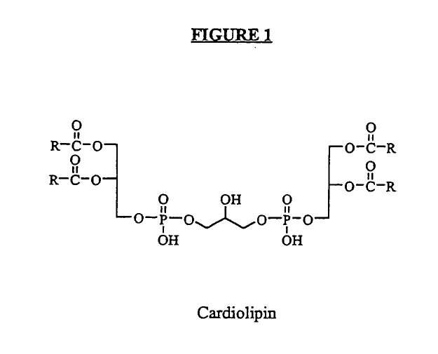

In the most preferred embodiment, Z₁ and Z₂ are —O—C(O)—, R₁ and R₂ are the same and are a C₄ to C₃₄ saturated and/or unsaturated alkyl group, more preferably having between 14 and 24 carbon atoms (such as between about 16 and about 20 carbon atoms). The term “alkyl” encompasses saturated or unsaturated straight-chain and branched-chain hydrocarbon moieties. The term “substituted alkyl” comprises alkyl groups further bearing one or more substituents selected from hydroxy, alkoxy (of a lower alkyl group), mercapto (of a lower alkyl group), cycloalkyl, substituted cycloalkyl, halogen, cyano, nitro, amino, amido, imino, thio, —C(O)H, acyl, oxyacyl, carboxyl, and the like. R₃ most preferably is CH₂. R₄ preferably is hydrogen. X most preferably is hydrogen or ammonium ion, which gives the general structure of cardiolipin as shown in FIG. 1.

Cardiolipin molecules and analogues can be prepared by any desired method. One preferred method provided by the instant invention for preparing a cardiolipin molecule or an analogue thereof involves reacting phosphatidic acid and 2-O-protected glycerol in the presence of a coupling agent, which is N,N′-dicyclohexylcarbodimide or N,N-carbonyldimidazole. Another preferred embodiment of the invention is a method for producing a cardiolipin or analogue thereof comprising reacting phosphatidic acid and glycerol in the presence of a coupling agent, which is triisopropylbenzenesulfonyl chloride, or N,N′-dicyclohexylcarbodiimide or N,N′-carbonyldimidazole.

A suitable method for preparing cardiolipin and its analogues, such as the inventive compounds, which method is an embodiment of the present invention, involves reacting an alcohol of the formula V 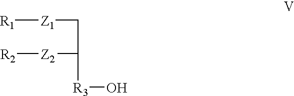
(wherein Z₁, Z₂, R₁, R₂ and R₃ are as defined above) and 2-O-protected glycerol or 2-O-subsituted glycerol in the presence of a coupling agent, which coupling agent is either dichlorophosphate or N,N-diisopropylmethylphosphonamidic chloride. This method is particularly suitable for preparing cardiolipins of formulas I, II, and III. Another embodiment of the inventive method, particularly suitable for preparing a cardiolipin of formula II involves reacting 1,2-O-diacyl glycerol and 2-O-protected glycerol in the presence of a coupling agent, which is either dichlorophosphate or N,N-diisopropylmethylphosphonamidic chloride. Another embodiment of the inventive method, particularly preferred for preparing the cardiolipin ether analogue of formula III involves reacting 1,2-O-dialkyl glycerol and 2-O-protected glycerol in the presence of a coupling agent, which is either dichlorophosphate or N,N-diisopropylmethylphosphonamidic chloride.

Another method for synthesizing cardiolipin molecules and analogues thereof, which method is an embodiment of the present invention, involves reacting an alcohol of formula V (above) and a diol of the formula VI 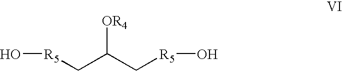
(wherein R₄ and R₅ are as defined above) in the presence of a coupling agent, which coupling agent is either dichlorophosphate or N,N-diisopropylmethylphosphonamidic chloride. This method is particularly suitable for producing molecules according to formula IV.

A preferred coupling agent for use in the synthetic methods of the invention is a dichlorophosphate of formula VII 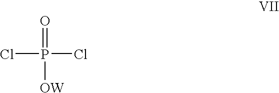
wherein W is alkyl groups or substituted alkyl groups including methyl, ethyl, isopropyl, t-butyl, allyl, 2-substituted ethyl, haloethyl such as 2,2,2-tribromoethyl; benzyl or substituted benzyl groups; phenyl or substituted phenyl groups such as 2-chlorophenyl, 4-chlorophenyl and 2,4-dichlorophenyl; or any other removable protecting groups. Another preferred coupling agent for use in the context of the inventive synthetic methods is N,N-diisopropylmethylphosphonamidic chloride.

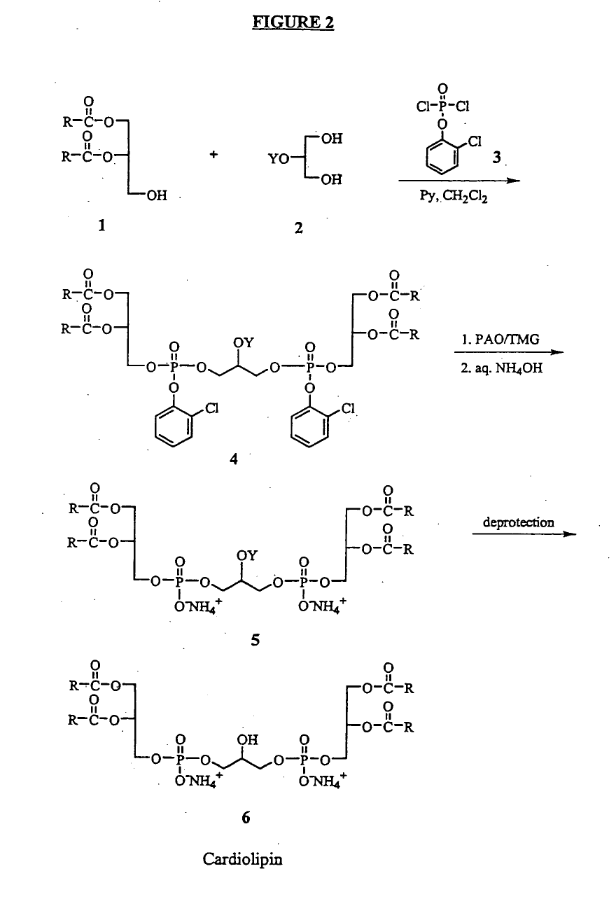

One embodiment of the present invention is set forth in FIG. 2, which depicts a novel approach to the synthesis of cardiolipin. In this method, a phosphorylating reagent, o-chlorophenyl dichlorophosphate (CPDCP) 3, is reacted with 1,2-O-diacyl glycerol 1 and 2-O-protected glycerol 2 (Y is a hydroxy protecting group, preferably a benzyl group or the like, or a silyl protecting group, for example, t-butyldimethylsilyl and the like) in an inert solvent (for example, dichloromethane and the like) in the presence of a base (for example, pyridine or the like) to provide cardiolipin precursor 4. The removal of the o-chlorophenyl can be accomplished by reaction of 4 with 2-pyridinealdoxime (PAO) and 1,1,3,3-tetramethylguanidine (TMG), followed by treatment with aqueous ammonium hydroxide, to provide a ammonium salt of cardiolipin precursor 5. Besides 2-pyridinealdoxime (PAO), other reagents such as 2-nitrobenzaldoxime in the presence of TMG can be used for the removal of o-chlorophenyl groups.

Deprotection to yield cardiolipin 6 can be accomplished by a method depending on the nature of the protecting group. For example, a benzyl group can be removed by hydrogenation in the presence of palladium catalyst. A t-butyldimethylsilyl (TBDMS) group can be deprotected under the acidic condition. A p-methoxybenzyl (PMB) group can be deprotected by treatment with 2,3-dichloro-5,6-dicyano-1,4-benzoquinone (DDQ).

The term “hydroxy protecting group” as used herein refers to groups used to protect hydroxy group against undesirable reactions during synthetic procedures. Commonly used hydroxy protecting groups are disclosed in T. W. Greene and P. G. M. Wuts, Protective Groups in Organic Synthesis, 3rd edition, John Wiley & Sons, New York (1999). Such hydroxy protecting groups include: methyl ether; substituted methyl ethers, including methoxymethyl, benzyloxymethyl, p-methoxybenzyloxymethyl, t-butoxymethyl, 2-methoxyethoxymethyl, tetrahydropyranyl, tetrahydrofuranyl ether and the like; substituted ethyl ethers, including, 1-ethoxyethyl, 1-methyl-1-methoxyethyl, 1-methyl-1-benzyloxyethyl, allyl, t-butyl ether and the like; benzyl ether; substituted benzyl ethers, including p-methoxybenzyl, 3,4-dimethoxybenzyl ether and the like; silyl ethers, including trimethylsilyl, triethylsilyl, dimethylisopropylsilyl, diethylisopropylsilyl, t-butyldimethylsilyl ether and the like; esters, including formate, acetate, chloroacetate, dichloroacetate, trichloroacetate, methoxyacetate, phenoxyacetate and the like; and carbonates.

The invention described above is a simple, efficient method to prepare cardiolipin. The key step of the synthesis is the phosphorylation coupling reaction. In this phosphotriester approach, CPDCP is used to sequentially phosphorylate alcohols in a straightforward manner to directly provide the intermediate phosphate triester in good yield. The method is a simple, cost effective one-pot process to build the cardiolipin core structure.

This novel dichlorophosphate coupling protocol of the invention is superior to the traditional phosphorodi(1,2,4-triazolide) or phosphorobis(hydroxybenzotriazole) approach (Ramirez et al., Synthesis, 449-489 (1985); van Boeckel et al., Tetrahedron Lett., 21:3705-3708 (1980); van Boeckel et al., Synthesis, 399-402 (1982) ). In one aspect, the phosphorodi(1,2,4-triazolide) or phosphorobis(hydroxybenzotriazole) needs to be prepared from the corresponding dichlorophosphare. In further aspect, the phosphorodi(1,2,4-triazolide) approach, in most case, preferably uses an additional activating reagent such as 2,4,6-triisopropylbenzenesulfonyl-(3-nitro-1,2,4-triazole) or 2,4,6-triisopropylbenzenesulphonyl chloride in the condensation reaction with the second alcohol.

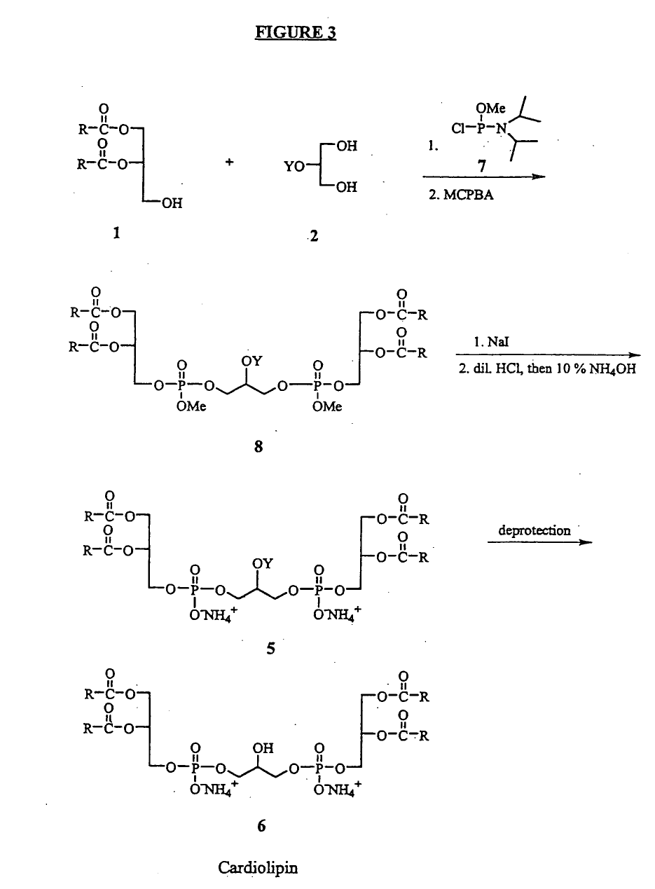

Another embodiment of the present invention, represented in FIG. 3, involves coupling of a 1,2-disubstituted glycerol with 2-protected glycerol using a novel phosphorylating agent: chlorophosphoramidite. (While N,N-diisopropylmethylphosphonamidic chloride 7 has been used in the synthesis of inositol phospholipids (Bruzik et al., Tetrahedron Lett., 36: 2415-2418 (1995), it has not been used in the synthesis of cardiolipin). In this method, chlorophosphoramidite 7 is used in the coupling reaction to build the core structure. In this scheme, 1,2-O-diacyl glycerol 1 is subsequently reacted with the reagent 7 in an inert solvent (for example, dichloromethane and the like) in the presence of a base (for example, N,N-diisopropylethylamine or the like), then with 2-O-protected glycerol 2 (Y is a hydroxy protecting group, preferably a benzyl group or the like) in the presence of an activator such as tetrazole or the like followed by oxidation with m-chloroperoxybenzoic acid (MCPBA) or the like to yield protected cardiolipin precursor 8. The methyl group of the protected precursor 8 then is removed by reaction with NaI to produce a sodium salt of a cardiolipin precursor, which is then converted to an ammonium salt 5 by treatment with dilute HCl followed by 10% ammonium hydroxide. Deprotection to yield cardiolipin can occur as described above.

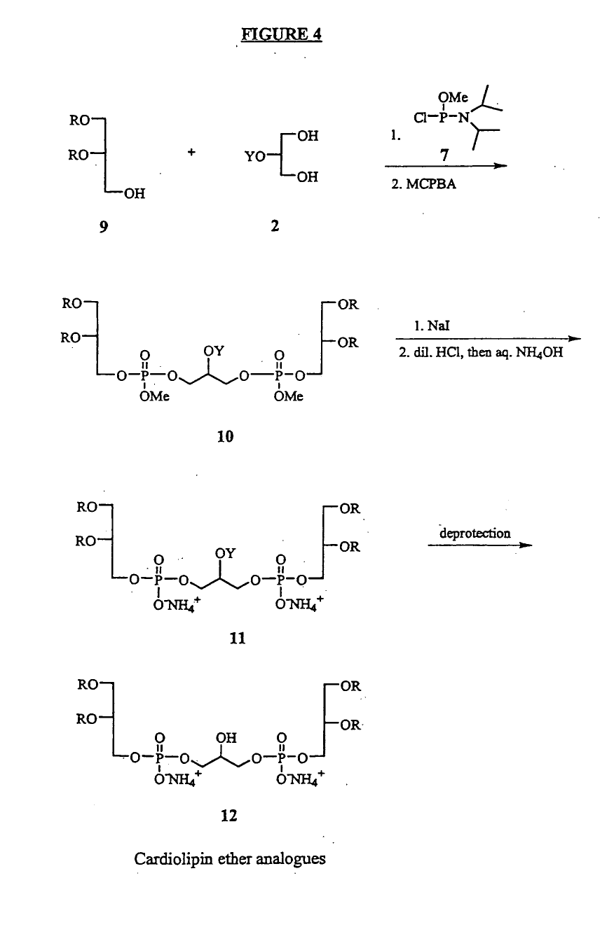

Another embodiment of the present invention, represented in FIG. 4, produces ether analogues of cardiolipin, wherein alkyl groups replace the acyl groups of the cardiolipin glycerol side chain. In accordance with this scheme, 1,2-O-dialkyl-sn-glycerol 9 is reacted to couple it with 2-O-protected glycerol 2 in the presence of chlorophosphoramidite 7 to provide an intermediate 10. With the similar procedure described in FIG. 3, demethylation of this intermediate 10 with NaI in 2-butanone yields the protected cardiolipin analogue 11, which on deprotection yields ether analogue 12 of cardiolipin.

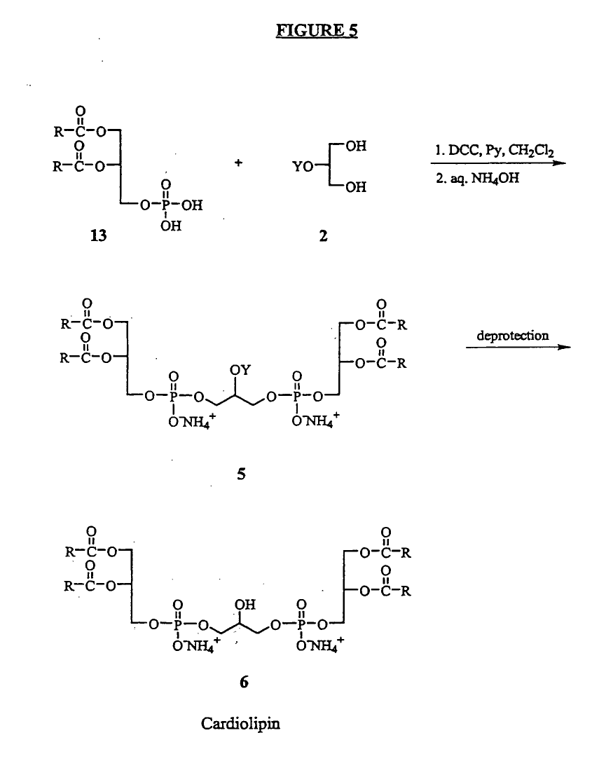

Another embodiment of the present invention is represented in FIG. 5. In this method, phosphatidic acid (PA) 13 is reacted with 2-O-protected glycerol 2 (Y is a hydroxy protecting group, preferably a benzyl group or the like, or a silyl protecting group, for example, t-butyldimethylsilyl and the like) in an inert solvent (for example, dichloromethane and the like) in the presence of a condensing reagent such as N,N′-dicyclohexylcarbodiimide (DCC), or N,N′-carbonyldiimidazole (CDI) or the like followed by treatment with aqueous ammonium hydroxide to form cardiolipin precursor 5. Deprotection yields cardiolipin 6 as described above.

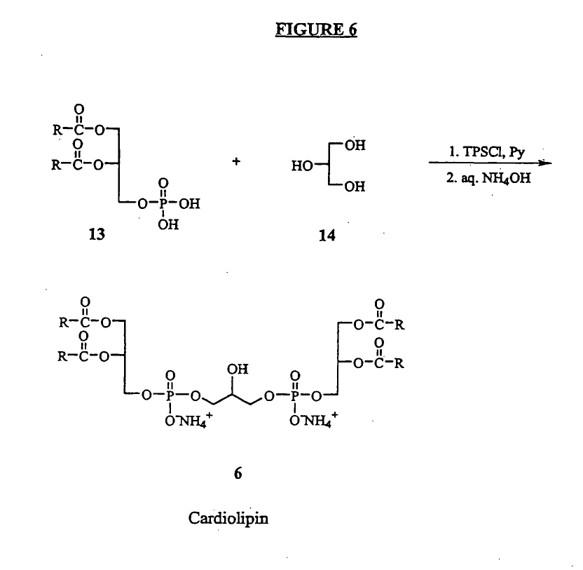

Another embodiment of the present invention is represented in FIG. 6. In this method, glycerol 14 without protecting group, is directly used as a reactant in the condensation reaction. Selective phosphorylation of the primary alcohol of glycerol 14 occurs by condensation with phosphatidic acid (PA) 13 in the presence of triisopropylbenzenesulfonyl chloride (TPSCI) and pyridine followed by treatment with aqueous ammonium hydroxide to yield cardiolipin 6. Other coupling reagents such as DCC, CDI or the like can also be used in this one step synthesis of cardiolipin.

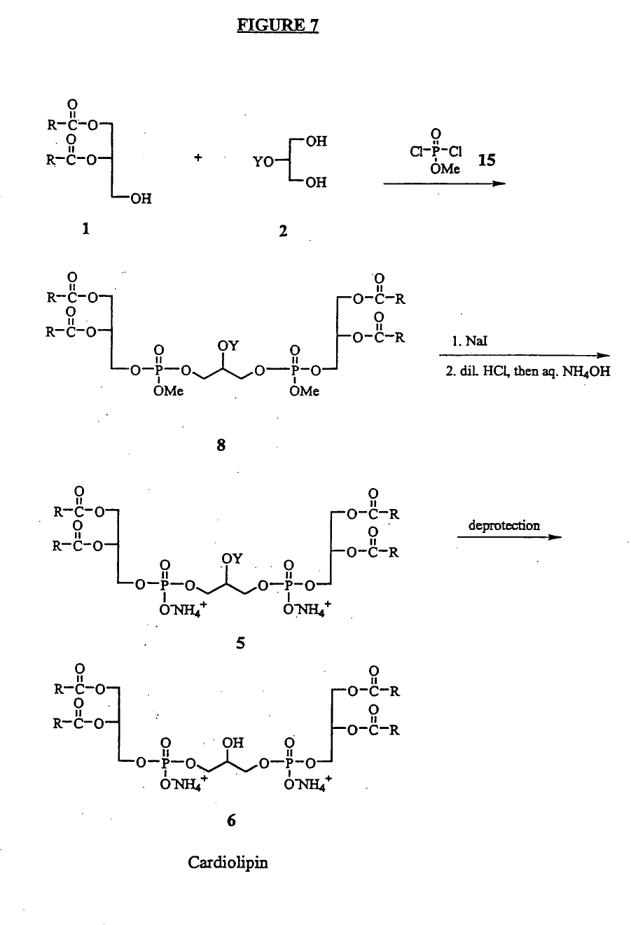

Another embodiment of the present invention is represented in FIG. 7. In this method, a dichlorophosphate 15 is used in a coupling reaction to build the core structure. In this scheme, 1,2-diacyl glycerol 1 and 2-protected glycerol 2 are reacted with the dichlorophosphate 15 in the presence of a base such as pyridine to yield a protected cardiolipin precursor 8. The methyl group of the protected cardiolipin 8 then is removed by reaction with NaI to produce a sodium salt of a cardiolipin precurser, when is then converted to an ammonium salt 5 by treatment with dilute HCl followed by 10% ammonium hydroxide. Deprotection to yield mature cardiolipin can occur as described above.

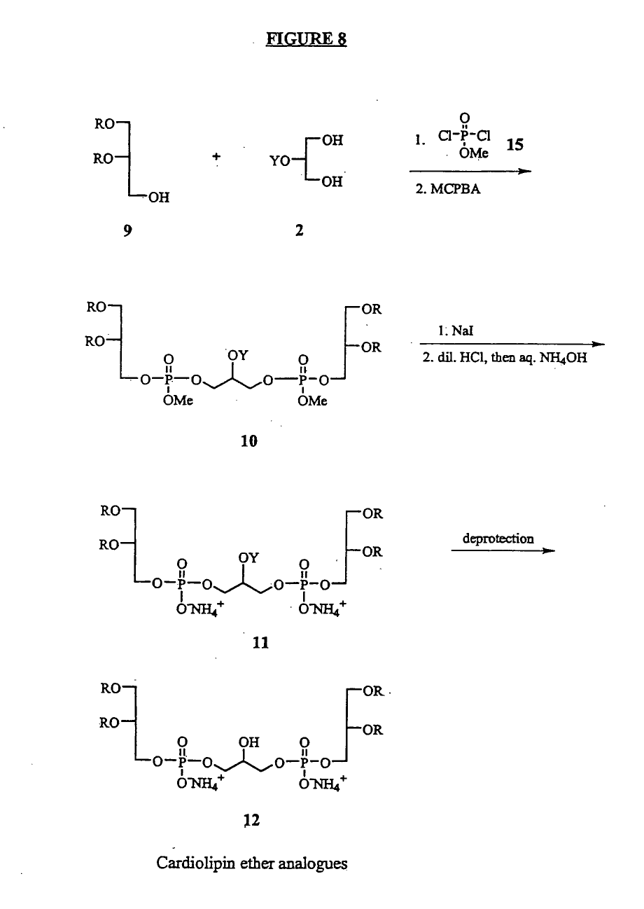

Another embodiment of the present invention is represented in FIG. 8. In this method, which results in ether analogues of cardiolipin, a dichlorophosphate 15 is used in a coupling reaction to build the core structure. In this scheme, 1,2-dialkyl-sn-glycerol 9 and 2-protected glycerol 2 are reacted with the dichlorophosphate 15 to yield a protected an intermediate 10. The methyl group of the intermediate 10 then is removed to produce a protected ammonium salt 11 by reaction with NaI and treatment with dilute HCl followed by 10% ammonium hydroxide. Deprotection to yield mature cardiolipin can occur as described above.

The described methods can be used to prepare a variety of novel cardiolipin species. For example, the methods can be used to prepare cardiolipin and analogues thereof containing any desired fatty acid chain as an R₁ and/or R₂ substituents. Preferred fatty acids range from carbon chain lengths of about C₄ to about C₃₄, preferably between about C₁₄ and about C₂₄, and include tetranoic acid (C4:0), pentanoic acid (C5:0), hexanoic acid (C6:0), heptanoic acid (C7:0), octanoic acid (C8:0), nonanoic acid (C9:0), decanoic acid (C10:0), undecanoic acid (C11:0), dodecanoic acid (C12:0), tridecanoic acid (13:0), tetradecanoic acid (C14:0), pentadecanoic acid (C15:0), hexadecanoic acid (C16:0), heptadecanoic acid (C17:0), octadecanoic acid (C18:0), nonadecanoic acid (C19:0), eicosanoic acid (C20:0), heneicosanoic acid (C21:0), docosanoic acid (C22:0), tricosanoic acid (C23:0), tetracosanoic acid (C24:0), 10-undecanoic acid (C11:1), 11-dodecanoic acid (C12:1), 12-tridecenoic acid (C13:1), myristoleic acid (C14:1), 10-pentadecenoic acid (C15:1), palmitoleic acid (C16:1), oleic acid (C18:1), linoleic acid (C18:2), linolenic acid (C18:3), eicosdienoic acid (C20:2), eicostrienoic acid (C20:3), arachidonic acid (cis-5,8,11,14-eicosatetraenoic acid), and cis-5,8,11,14,17-eicosapentaenoic acid, among others. For ether analogues, the alkyl chain also will range from carbon chain lengths of about C₄ to about C₃₄, preferably between about C₁₄ and about C₂₄. Other fatty acid chains also can be employed as R₁ and/or R₂ sustituents. Examples of such include saturated fatty acids such as methanoic (or formic) acid, ethanoic (or acetic) acid, propanoic (or proprionic) acid, butanoic (or butyric) acid, hexacosanoic (or cerotic) acid, octacosanoic (or montanic) acid, triacontanoic (or melissic) acid, dotriacontanoic (or lacceroic) acid, tetratriacontanoic (or gheddic) acid, pentantriacontanoic (or ceroplastic) acid, and the like; monoethenoic unsaturated fatty acids such as trans-2-butenoic (or crotonic) acid, cis-2-butenoic (or isocrotenoic) acid, 2-hexenoic (or isohydrosorbic) acid, 4-decanoic (or obtusilic) acid, 9-decanoic (or caproleic) acid, 4-dodecenoic (or linderic) acid, 5-dodecenoic (or denticetic) acid, 9-dodecenoic (or lauroleic) acid, 4-tetradecenoic (or tsuzuic) acid, 5-tetradecenoic (or physeteric) acid, 6-octadecenoic (or petroselinic) acid, trans-9-octadecenoic (or elaidic) acid, trans-11-octadecenoic (or vaccinic) acid, 9-eicosenoic (or gadoleic) acid, 11-eicosenoic (or gondoic) acid, 11-docosenoic (or cetoleic) acid, 13-decosenoic (or erucic) acid, 15-tetracosenoic (or nervonic) acid, 17-hexacosenoic (or ximenic) acid, 21-triacontenoic (or lumequeic) acid, and the like; dienoic unsaturated fatty acids such as 2,4-pentadienoic (or β-vinylacrylic) acid, 2,4-hexadienoic (or sorbic) acid, 2,4-decadienoic (or stillingic) acid, 2,4-dodecadienoic acid, 9,12-hexadecadienoic acid, cis-9,cis-12-octadecadienoic (or α-linoleic) acid, trans-9,trans-12-octadecadienoic (or linlolelaidic) acid, trans-10-trans-12-octadecadienoic acid, 11,14-eicosadienoic acid, 13,16-docosadienoic acid, 17,20-hexacosadienoic acid, and the like; trienoic unsaturated fatty acids, such as 6,10,14-hexadecatrienoic (or hiragonic) acid, 7,10,13-hexadecatrienoic acid, cis-6,cis-9,cis-12-octadecatrianoic (or γ-linolenic) acid, trans-8,trans,10,cis-12-octadecatrienoic (or α-calendic) acid, trans-8,trans-10,trans-12 octadecatrienoic (or β-calendic) acid, cis-8,trans-10,cis-12-octadecatrienoic acid, cis-9,cis-12,cis-15-octadecatrienoic (or α-linolenic) acid, trans-9,trans-12,trans-15-octadecatrienoic (or linolenelaidic) acid, cis-9,trans-11,trans-13-octadecatrienoic (or α-eleostearic) acid, trans-9,trans-11,trans-13-octadecatrienoic (or β-eleostearic) acid, cis-9,trans-11,cis-13-octadecatrienoic (or punicic) acid, trans-9,trans-11,trans-13-octadecatrienoic acid, 5,8,11-eicosatrienoic acid, 8,11,14-eicosatrienoic acid, and the like; tetraenoic unsaturated fatty acids such as 4,8,11,14-hexadecatetraenoic acid, 6,9,12,15-hexadecatetraenoic acid, 4,8,12,15-octadecatetraenoic (or moroctic) acid, 6,9,12,15-octadecatetraenoic acid, 9,11,13,15-octadecatetraenoic (or α- or β-parinaric) acid, 9,12,15,18-octadecatetraenoic acid, 4,8,12,16-eicosatetraenoic acid, 6,10,14,18-eicosatetraenoic acid, 4,7,10,13-docosatetraenoic acid, 7,10,13,16-docosatetraenoic acid, 8,12,16,19-docosatetraenoic acid, and the like; penta- and hexa-enoic unsaturated fatty acids such as 4,8,12,15,18-eicosapentaenoic (or timnodonic) acid, 4,7,10,13,16-docosapentaenoic acid, 4,8,12,15,19-docosapentaenoic (or clupanodonic) acid, 7,10,13,16,19-docosapentaenoic acid, 4,7,10,13,16,19-docosahexaenoic acid, 4,8,12,15,18,21-tetracosahexaenoic (or nisinic) acid, and the like; branched-chain fatty acids such as 3-methylbutanoic (or isovaleric) acid, d-6-methyloctanoic acid, 8-methyldecanoic acid, 10-methylundecanoic (or isolauric) acid, d-10-methyldodecanoic acid, 11-methyldodecanoic (or isoundecylic) acid, 12-methyltridecanoic (or isomyristic) acid, d-12-methyltetradecanoic acid, 13-methyltetradecanoic (or isopentadecylic) acid, 14-methylpentadecanoic (or isopalmitic) acid, d-14-methylhexadecanoic acid, 15-methylhexadecanoic acid, 10-methylheptadecanoic acid, 16-methylheptadecanoic (or isostearic) acid, t-D-10-methyloctadecanoic (or tuberculostearic) acid, d-16-methyloctadecanoic acid, 18-methylnonadecanoic (or isoarachidic) acid, d-18-methyleicosanoic acid, 20-methylheneicosanoic (or isobehenic) acid, d-20-methyldocosanoic acid, 22-methyltricosanoic (or isolignoceric) acid, d-22-methyltetracosanoic acid, 24-methylpentacosanoic (or isocerotic) acid, d-24-methylhexacosanoic acid, 26-methylheptacosanoic (or isomonatanic) acid, d-28-methyltriacontanoic acid, 2,4,6-(D)-trimethyloctacosanoic (or mycoceranic or mycoserosic) acid, 2-methyl-cis-2-butenoic (angelic) acid, 2-methyl-trans-2-butenoic (or tiglic) acid, 4-methyl-3-pentenoic (or pyroterebic) acid, d-2,4(L),6(L)-trimethyl-trans-2-tetracosenoic (or C₂₇-phthienoic or mycolipenic) acid, and the like.

The cardiolipin molecules described herein, and cardiolipin molecules produced by the inventive method can be used in lipid formulations. A preferred formulation or composition is a liposomal composition including the inventive cardiolipin analogues. Complexes, emulsions, and other formulations including the inventive cardiolipin also are within the scope of the present invention. Such formulations according to the present invention can be prepared by any suitable technique. In addition to the inventive synthetic cardiolipin, the liposomal composition, complex, emulsion, and the like can include stabilizers, absorption enhancers, antioxidants, phospholipids, biodegradable polymers, and medicinally active agents among other ingredients. In some embodiments, it is preferable for the inventive composition, especially a liposomal composition, also to include a targeting agent, such as a carbohydrate or a protein or other ligand that binds to a specific substrate, such as antibodies (or fragments thereof) or ligands that recognize cellular receptors. The inclusion of such agents (such as a carbohydrate or one or more proteins selected from groups of proteins consisting of antibodies, antibody fragments, peptides, peptide hormones, receptor ligands, such as an antibody to a cellular receptor, and mixtures thereof) can facilitate targeting a liposome to a predetermined tissue or cell type.

Lipophilic liposome-forming ingredients, such as phosphatidylcholine, a cardiolipin prepared by the methods described above, cholesterol and α-tocopherol can be dissolved or dispersed in a suitable solvent or combination of solvents and dried. Suitable solvents include any non-polar or slightly polar solvent, such as t-butanol, ethanol, methanol, chloroform, or acetone that can be evaporated without leaving a pharmaceutically unacceptable residue. Drying can be by any suitable means such as by lyophilization, and it is preferred also to employ a cryoprotectant (e.g., a protective sugar such as trehalose) during lyophilization. Hydrophilic ingredients can be dissolved in polar solvents, including water.

Liposomes can be formed by mixing the dried lipophilic ingredients with the hydrophilic mixture. Mixing the polar solution with the dry lipid film can be by any means that strongly homogenizes the mixture. The homogenization can be effected by vortexing, magnetic stirring and/or sonicating.

Liposomes also can contain active agents, and the invention provides a method of retaining an active agent in a liposome. The method invovles preparing a cardiolipin or cardiolipin analogue as described herein, and including the cardiolipin or cardiolipin analogue and an active agent in a liposome. The active agent can become complexed with a portion of the lipid (such as the inventive cardiolipin), or the active agent can become entrapped within the liposomes. In accordance with the method, the active agents can be dissolved or dispersed in a suitable solvent and added to the liposome mixture prior to mixing. Typically hydrophilic active agents will be added directly to the polar solvent and hydrophobic active agents will be added to the nonpolar solvent used to dissolve the other ingredients but this is not required. The active agent could be dissolved in a third solvent or solvent mix and added to the mixture of polar solvent with the lipid film prior to homogenizing the mixture.

Generally, liposomes can have net neutral, negative or positive charge. For example, positive liposomes can be formed from a solution containing phosphatidylcholine, cholesterol, cardiolipin and enough stearylamine to overcome the net negative charge of cardiolipin. Negative liposomes can be formed from solutions containing phosphatidylcholine, cholesterol, and/or cardiolipin, for example.

Liposomes including the inventive cardiolipin also can include other constituents within the lipid phase. Preferred constituents include a phosphatidylcholine selected from the group consisting of dimyristoylphosphatidylcholine, distearoylphosphatidylcholine, dioleylphosphatidylcholine, dipalmitoylphosphatidylcholine, diarachidonoylphosphatidylcholine, egg phosphatidylcholine, soy phosphatidylcholine, hydrogenated soy phosphatidylcholine, and mixtures thereof. Another preferred constituent is a phosphatidylglycerol, selected from the group consisting of dimyristoylphosphatidylglycerol, distearoylphosphatidylglycerol, dioleylphosphatidylglycerol, dipalmitoylphosphatidylglycerol, diarachidonoylphosphatidylglycerol, and mixtures thereof. The liposomes also can include a sterol selected from the group consisting of cholesterol, polyethylene glycol, derivatives of cholesterol, coprostanol, cholestanol, cholestane, cholesterol hemisuccinate, cholesterol sulfate, and mixtures thereof.

The liposomes of the present invention can be multi or unilamellar vesicles depending on the particular composition and procedure used to make them. Liposomes can be prepared to have substantially homogeneous sizes in a selected size range, such as about 1 micron or less, or about 500 nm or less, about 200 nm or less, or about 100 nm or less. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane.

Liposomes can be coated with a biodegradable polymers such as sucrose, epichlorohydrin, branched hydrophilic polymers of sucrose, polyethylene glycols, polyvinyl alcohols, methoxypolyethylene glycol, ethoxypolyethylene glycol, polyethylene oxide, polyoxyethylene, polyoxypropylene, cellulose acetate, sodium alginate, N,N-diethylaminoacetate, block copolymers of polyoxyethylene and polyoxypropylene, polyvinyl pyrrolidone, polyoxyethylene X-lauryl ether wherein X is from 9 to 20, and polyoxyethylene sorbitan esters.

Antioxidants can be included in liposome formulations, complex, emulsion, or other formulations of the present invention. Suitable antioxidants include compounds such as ascorbic acid, tocopherol, and deteroxime mesylate.

Absorption enhancers also can be included in the inventive liposomal formulations, complexes, emulsions, and the like, if desired. Suitable absorption enhancers include Na-salicylate-chenodeoxy cholate, Na-deoxycholate, polyoxyethylene 9-lauryl ether, chenodeoxy cholate-deoxycholate and polyoxyethylene 9-lauryl ether, monoolein, Na-tauro-24,25-dihydrofusidate, Na-taurodeoxycholate, Na-glycochenodeoxycholate, oleic acid, linoleic acid, linolenic acid. Polymeric absorption enhancers can also be included such as polyoxyethylene ethers, polyoxyethylene sorbitan esters, polyoxyethylene 10-lauryl ether, polyoxyethylene 16-lauryl ether, azone (1-dodecylazacycloheptane-2-one).

The liposomal and other types of compositions of the invention, including the inventive cardiolipin (including those prepared in accordance with the inventive method) can be formulated to include active agents. The active agent can be, for example, entrapped within liposomes within the composition, complexed with one of the ingredients in the composition (e.g., complexed with the cardiolipin within the composition), or otherwise present within the composition. Inclusion within the inventive composition is thought to be general for active agents that are stable in the presence of surfactants. Where the composition includes liposomes, hydrophilic active agents are suitable and can be included in the interior of liposomes such that the liposome bilayer creates a diffusion barrier preventing it from randomly diffusing throughout the body. Hydrophobic active agents are thought to be particularly well suited for use in the inventive formulations, particularly liposomal formulations, because they not only benefit by exhibiting reduced toxicity but they tend to be well solubilized in the lipid bilayer of liposomes. For medical or cosmetic use, the formulation can be physiologically compatible, such as pharmaceutically acceptable, and can include other agents (e.g., buffers, antibiotics, preservatives and other excipients, such as one or more pharmaceutically acceptable excipients) known to those of ordinary skill for formulating pharmaceutical compositions.

Where a composition according to the invention contains active agents, the invention provides also a method of using such formulations to administer active agents to human or animal cells. The cells can be in vitro, in which case the formulations can be used for diagnostic or investigative purposes, or for delivering active agents to cells to be implanted into a human or animal patient. Alternatively, the cells can be in vivo, in which instance, the invention provides a method for delivering active agents into human or animals, for example patients in need of therapy using the active agent or for cosmetic purposes. The method can be used to administer virtually any active agent, for example into diseased cells or organs of patients. Suitable active agents include diagnostic reagents and pharmaceutical agents used to treat disorders such as inflammation (e.g., chronic inflammation), angiogenesis-dependent diseases, arthritis, restenosis, psoriasis, cancer (e.g., lung cancer, brain cancer or other cancers of the central nervous system, melanoma, pancreatic cancer, liver cancer, cancers of the testes or ovaries, and other neoplastic disorders), multiple sclerosis, alzheimers, parkinsons, and a variety of vascular diseases. Liposomal formulations, emulsion, or complexes also can be useful for anti-fungal application, and can contain suitable anti-fungals as active agents. Typically, active agents can be one or more genes and gene vectors, antisense molecules (e.g., oligonucleotides), proteins and peptides, protein or chemical drugs (e.g., hydrophobic or hydrophilic drugs) or diagnostic agents.

Active agents which are compatible with the present invention include agents which act on the peripheral nerves, adrenergic receptors, cholinergic receptors, the skeletal muscles, the cardiovascular system, smooth muscles, the blood circulatory system, synaptic sites, neuroeffector junctional sites, endocrine and hormone systems, the immunological system, the reproductive system, the skeletal system, the alimentary and excretory systems, the histamine system and the central nervous system. Suitable agents may be selected from, for example, proteins, enzymes, hormones, nucleotides, polynucleotides, nucleoproteins, polysaccharides, glycoproteins, lipoproteins, polypeptides, steroids, terpenoids, triterpines, retinoids, anti-ulcer H2 receptor antagonists, antiulcer drugs, hypocalcemic agents, moisturizers, cosmetics, etc. Active agents can be analgesics; anesthetics; anti-arrythmic agents, antibiotics; antiallergic agents, antifungal agents, anticancer agents (e.g., mitoxantrone (see, e.g., international patent publication WO 02/32400), taxanes (see, e.g., international patent publication WO 00/01366), paclitaxel, camptothecin, and camptothecin derivaties ((e.g., SN-38) (see, e.g., international patent publication WO 02/058622), irinotecan (see, e.g., international patent publication WO 03/030864), and other camptothecins), gemcitabine, anthacyclines, antisense oligonucleotides (e.g., targeting oncogenes, such as a raf gene (see. e.g., international patent publication WO 98/43095)), vinca alkaloids (e.g., vinorelbine, see, e.g, international patent publication WO 03/018018)), antibodies, cytoxines, immunotoxines, etc.), antihypertensive agents (e.g., dihydropyridines, antidepressants, cox-2 inhibitors); anticoagulants; antidepressants; antidiabetic agents, anti-epilepsy agents, antiinflammatory corticosteroids; agents for treating Alzheimers or Parkinson's disease; antiulcer agents; anti-protozoal agents, anxiolytics, thyroids, anti-thyroids, antivirals, anoretics, bisphosphonates, cardiac inotropic agents, cardiovascular agents, corticosteroids, diuretics, dopaminergic agents, gastrointestinal agents, hemostatics, hypercholesterol agents, antihypertensive agents; immunosuppressive agents; anti-gout agents, anti-malarials, anti-migraine agents, antimuscarinic agents, antiinflammatory agents, such as agents for treating rheumatology, arthritis, psoriasis, inflammatory bowel disease, Crohn's disease; or agents for treating demyelinating diseases including multiple sclerosis; ophthalmic agents; vaccines (e.g., against influenza virus, pneumonia, hepatitis A, hepatitis B, hepatitis C, cholera toxin B-subunit, typhoid, plasmodium falciparum, diptheria, tetanus, herpes simplex virus, tuberculosis, HIV, SARS virus, bordetela pertusis, measules, mumps, rubella, bacterial toxoids, vaccinea virus, adenovirus, canary virus, bacillus calmette Guerin, klebsiella pneumonia vaccine, etc.); histamine receptor antagonists, hypnotics, kidney protective agents, lipid regulating agents, muscle relaxants, neuroleptics, neurotropic agents, opioid agonists and antagonists, parasympathomimetics, protease inhibitors, prostglandins, sedatives, sex hormones (e.g., androgens, estrogens, etc.), stimulants, sympathomimetics, vasodilators and xanthins and synthetic analogues of these species. The therapeutic agents can be nephrotoxic, such as cyclosporins and amphotericin B, or cardiotoxic, such as amphotericin B and paclitaxel. Exemplary anticancer agents include melphalan, chlormethine, extramustinephosphate, uramustine, ifosfamide, mannomustine, trifosfamide, streptozotocin, mitobronitol, mitoxantrone, methotrexate, fluorouracil, cytarabine, tegafur, idoxide, taxol, paclitaxel, daunomycin, daunorubicin, bleomycin, amphotericin, carboplatin, cisplatin, paclitaxel, BCNU, vincristine, camptothecin, doxorubicin, etopside, cytokines, ribozymes, interferons, oligonucleotides and functional derivatives of the foregoing. Additional examples of drugs which may be delivered according to the method include, prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperzine maleate, anisindone, diphenadione erythrityl tetranitrate, digoxin, isoflurophate, acetazolamide, methazolamide, bendroflumethiazide, chloropromaide, tolazamide, chlormadinone acetate, phenaglycodol, allopurinol, aluminum aspirin, methotrexate, acetyl sulfisoxazole, erythromycin, hydrocortisone, hydrocorticosterone acetate, cortisone acetate, dexamethasone and its derivatives such as betamethasone, triamcinolone, methyltestosterone, 17-S-estradiol, ethinyl estradiol, ethinyl estradiol 3-methyl ether, prednisolone, 17α-hydroxyprogesterone acetate, 19-norprogesterone, norgestrel, norethindrone, norethisterone, norethiederone, progesterone, norgesterone, norethynodrel, aspirin, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, chlorpromazine, methyldopa, dihydroxyphenylalanine, theophylline, calcium gluconate, ketoprofen, ibuprofen, cephalexin, erythromycin, haloperidol, zomepirac, ferrous lactate, vincamine, diazepam, phenoxybenzamine, diltiazem, milrinone, mandol, quanbenz, hydrochlorothiazide, ranitidine, flurbiprofen, fenufen, fluprofen, tolmetin, alclofenac, mefenamic, flufenamic, difuinal, nimodipine, nitrendipine, nisoldipine, nicardipine, felodipine, lidoflazine, tiapamil, gallopamil, amlodipine, mioflazine, lisinolpril, enalapril, enalaprilat captopril, ramipril, famotidine, nizatidine, sucralfate, etintidine, tetratolol, minoxidil, chlordiazepoxide, diazepam, amitriptyline, and imipramine. Further examples are proteins and peptides which include, but are not limited to, bone morphogenic proteins, insulin, heparin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, gonadotropin releasing hormone, somatotropins (e.g., bovine somatotropin, porcine somatotropin, etc.), oxytocin, vasopressin, GRF, somatostatin, lypressin, pancreozymin, luteinizing hormone, LHRH, LHRH agonists and antagonists, leuprolide, interferons (e.g., α-, β-, or γ-interferon, interferon α-2a, interferon α-2b, and consensus interferon, etc.), interleukins, growth hormones (e.g., human growth hormone and its derivatives such as methione-human growth hormone and des-phenylalanine human growth hormone, bovine growth hormone, porcine growth hormone, insulin-like growth hormone, etc.), digestive hormones, thyroids, anti-thyroids, fertility inhibitors such as the prostaglandins, fertility promoters, growth factors such as insulin-like growth factor, coagulation factors, pancreas hormone releasing factor, analogues and derivatives of these compounds, and pharmaceutically acceptable salts of these compounds, or their analogues or derivatives. An active agent (for diagnostic or theraperutic use) also can be or include a nucleic acid, such as RNA, DNA (e.g., oligonucleotides, plasmids, phage or viral vectors, and the like). The active agent can be a mixture of agents (e.g., two or more) that can be beneficially co-administered in the liposome formulation, complex, emulsion, or other formulation.

Chemotherapeutic agents and other anticancer agents, such as noted above, are well suited for use in the inventive composition and method of treatment. Liposome formulations, complexes, emulsions, and the like containing chemotherapeutic and other anticancer agents may be injected directly into a tumor tissue for delivery of the chemotherapeutic and other anticancer agent directly to cancer cells. In some cases, particularly after resection of a tumor, the liposomal formulation, emulsion, complex, or other inventive formulation can be implanted directly into the resulting cavity or may be applied to the remaining tissue as a coating. In cases in which an inventive liposome formulation is administered after surgery, it is possible to utilize liposomes having larger diameters of about 1 micron since they do not have to pass through the vasculature.

Where the composition includes an active agent, the invention provides a method of delivering the active agent to a cell. In accordance with this method, a composition containing the inventive cardiolipin and a desired active agent is prepared as described herein. The composition then is exposed to the cell to as to deliver the active agent to the cell. The method can be used to deliver active agents such as drugs, nucleic acids, and other suitable agents to any desired cell. For example, the method can be used for in vitro applications to deliver active agents to cells in culture. Alternatively, the method can be used to deliver active agents, such as drugs to cells in vivo. Where the method is employed in vivo, it can be used to treat a human or animal disease. In this embodiment, the composition is exposed to the human or animal so as to deliver the active agent to the human or animal. In some applications, the agent and the composition can be used cosmetically. A preferred application of this method involves the treatment of cancers, such as where the composition contains one or more anti-cancer agents, as described herein.

For application in vivo, it is preferred for the composition to include one or more pharmaceutically acceptable excipients. Also, pharmaceutically active agents, such as anticancer drugs, nucleic acid and proteinaceous agents described herein, can be incorporated into the inventive compositions at a concentration suitable to deliver a pharmaceutically-effective dosage. The dosage of pharmaceutically active agents, such as anticancer agents, can be varied as deemed appropriate by the treating physician or veterinarian, and it is within the skill of such practitioners to select a suitable dosage for therapeutic treatment.

The method provides for the administration of pharmaceutical preparations which in addition to liposomal formulations of active agents (and other formulations containing the inventive cardiolipin and active agents) include non-toxic, inert pharmaceutically suitable excipients. Pharmaceutically suitable excipients include solid, semi-solid or liquid diluents, fillers and formulation auxiliaries of all kinds. The invention also includes pharmaceutical preparations in dosage units. This means that the preparations are in the form of individual parts, for example vials, syringes, capsules, pills, suppositories, or ampoules, of which the content of the liposome formulation of active agent corresponds to a fraction or a multiple of an individual dose. The dosage units can contain, for example, 1, 2, 3, or 4 individual doses, or ½, ⅓, or ¼ of an individual dose. An individual dose preferably contains the amount of active agent which is given in one administration and which usually corresponds to a whole, a half, a third, or a quarter of a daily dose.

Tablets, dragees, capsules, pills, granules, suppositories, solutions, suspensions and emulsions, pastes, ointments (e.g., dry skin ointments), gels, creams, lotions (such as dry skin softeners, moisturizers, and the like), powders and sprays can be suitable pharmaceutical preparations. Suppositories can contain, in addition to the liposomal active agent, suitable water-soluble or water-insoluble excipients. Suitable excipients are those in which the inventive liposomal active agent is sufficiently stable to allow for therapeutic use, for example polyethylene glycols, certain fats, and esters or mixtures of these substances. Ointments, pastes, creams and gels can also contain suitable excipients in which the liposomal active agent is stable.

The active agent or its pharmaceutical preparations can be administered intravenously, subcutaneously, locally, orally, parenterally, intraperitoneally, and/or rectally or by direct injection into tumors or organs or other sites in need of treatment by such methods as are known or developed. Cardiolipin and cardiolipin-analogue-based formulations also can be administered topically, e.g., as a cream, skin ointment, dry skin softener, moisturizer, etc.

The following examples further illustrate the invention, without limitation.

### EXAMPLE 1

### A. Synthesis of Fully Protected Cardiolipin

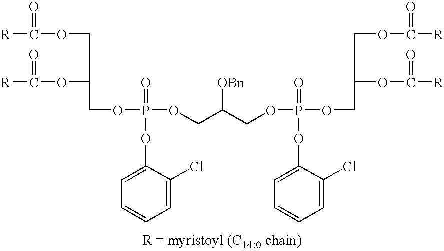

To a solution of o-chlorophenyl dichlorophosphate (2.45 g, 9.98 mmol) and dry pyridine (4.39 mL, 54.28 mmol) in CH₂Cl₂ (10 mL) was added dropwise a solution of 1,2-O-dimyristoyl-sn-glycerol (5.00 g, 9.75 mmol) in CH₂Cl₂ (50 mL) at 0° C. over 45 min. After the reaction mixture was stirred at 0° C. for 1 h and at rt for 1 h, a solution of 2-benzyloxy-1,3-propanediol (0.71 g, 3.90 mmol) in CH₂Cl₂ (8 mL) was added dropwise. The reaction mixture was stirred at rt for 3 h. The organic solvent was removed in vacuo and the residue was partitioned between ethyl acetate (150 mL) and cold 0.5N HCl (100 mL). The organic phase was washed with water, brine, dried over anhydrous Na₂SO₄ and concentrated in vacuo. The obtained residue was purified by flash chromatography on silica gel using hexane/ethyl acetate (3:1) to afford 4.37 g of fully protected cardiolipin as a colorless oil. The yield is 72%. TLC (Hexane/EtOAc 3:1) R_{f}=0.31; ¹HNMR (500 MHz, CDCl₃) δ 7.40-7.08 (m, 13H, ArH), 5.22 (m, 2H, RCOOCH), 4.63 (m, 2H, CH₂Ph), 4.40-4.06 (m, 12H, RCOOCH₂, POCH₂), 3.89 (m, 1H, BnOCH), 2.26 (m, 8H, —CH₂COO—), 1.57 (m, 8H, —CH₂CH₂COO—), 1.25 (br s, 80H, CH₂), 0.88 (t, J=6.5, 12H, CH₃); ESI-MS, m/z (M+Na)⁺ 1576.6.

### 1B. Synthesis of 2-O-Benzyl-13-bis(1,2-O-dimyristoyl-sn-glycero-3-phosphoryl)glycerol Diammonium Salt

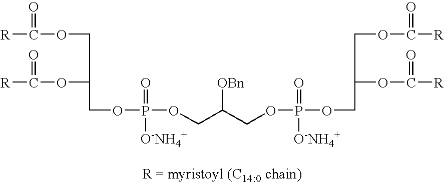

Method 1. To a stirred solution of fully protected cardiolipin (260.3 mg, 0.17 mmol) in THF (5 mL) was added 2-pyridinealdoxime (202.5 mg, 1.66 mmol) and tetramethylguanidine (176.0 mg, 1.53 mmol). After addition of 1 drop of water, the mixture was stirred at rt for 2.5 h. Solvent was removed in vacuo. The residue was dissolved in CHCl₃ (10 mL) and washed with H₂O (4 mL×2), dried over Na₂SO₄ and concentrated in vacuo. The obtained residue was purified by flash chromatography on silica gel using CHCl₃/MeOH/NH₄OH (65:15:1) to afford 200 mg of 2-O-benzyl-1,3-bis(1,2-O-dimyristoyl-sn-glycero-3-phosphoryl)glycerol diammonium salt as a white solid. The yield was 87%. TLC (CHCl₃/MeOH/NH₄OH 65:25:5) R_{f}=0.64; ¹HNMR (500 MHz, CDCl₃) δ 7.42-7.23 (m, 5H, ArH), 5.20 (m, 2H, RCOOCH), 4.60 (s, 2H, CH₂Ph), 4.29-3.89 (m, 12H, RCOOCH₂, POCH₂), 3.69 (m, 1H, BnOCH), 2.27 (m, 8H, —CH₂COO—), 1.56 (m, 8H, —CH₂CH₂COO—), 1.28 (br s, 80H, CH₂), 0.88 (t, J=6.5, 12H, CH₃); ESI-MS, m/z (M-2NH₄)²⁻664.9, (M-2NH₄—RCOO)⁻ 1102.0, (M-2NH₄+H)⁻ 1330.3.

Method 2. To a stirred solution of fully protected cardiolipin (3.88 g, 2.50 mmol) in THF (65 mL) was added 2-nitrobenzaldoxime (4.11 g, 24.74 mmol) and tetramethylguanidine (2.62 g, 22.75 mmol). After addition of 15 drops of water, the mixture was stirred at rt for 4 h. Solvent was removed in vacuo. The residue was dissolved in CHCl₃ (100 mL) and washed with H₂O (40 mL) and MeOH (2 mL). The organic phase was dried over Na₂SO₄ and concentrated in vacuo. The obtained yellow residue was purified by flash chromatography on silica gel using CHCl₃/MeOH/NH₄OH (65:15:1) to afford 2.17 g of 2-O-benzyl-1,3-bis(1,2-O-dimyristoyl-sn-glycero-3-phosphoryl)glycerol diammonium salt as a white solid. The yield is 64%. TLC (CHCl₃/MeOH/NH₄OH 65:25:5) R_{f}=0.64.

### 1C. Synthesis of 1,3-Bis(1,2-O-dimyristoyl-sn-glycero-3-phosphoryl)glycerol Diammonium Salt (Tetramyristoyl Cardiolipin)

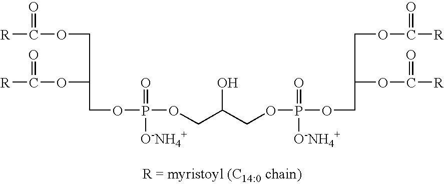

Method 1. A sample of 2-O-benzyl-1,3-bis(1,2-O-dimyristoyl-sn-glycero-3-phosphoryl)glycerol diammonium salt (520.1 mg, 0.38 mmol) was dissolved in THF (25 mL) and hydrogenated with palladium black (200 mg) for 3.5 h under a hydrogen balloon. After filtration to remove the catalyst, the solution was evaporated to dryness. The residue was dissolved in THF (7 mL), then precipitated using acetone (35 mL). The mixture was kept in freezer overnight and next day, the white solid was filtered and washed with a small amount of cold acetone. After drying in a vacuum desiccator under drierite for 12 h and under P₂O₅ for 5 h, 415.1 mg of 1,3-bis(1,2-O-dimyristoyl-sn-glycero-3-phosphoryl)glycerol diammonium salt (tetramyristoyl cardiolipin) was obtained. The yield is 86%. TLC (CHCl₃/MeOH NH₄OH 65:25:5) R_{f}=0.29; ¹HNMR (500 MHz, CDCl₃) δ 7.32 (br s, NH₄), 5.26 (m, 2H, RCOOCH), 4.34-3.92 (m, 13H, RCOOCH₂, POCH₂, HOCH), 2.33 (m, 8H, —CH₂COO—), 2.29 (t, J=7.5, 1H, CHOH), 1.58 (m, 8H, —CH₂CH₂COO—), 1.30 (br s, 80H, CH₂), 0.88 (t, J=6.5, 12H, CH₃); FTIR (ATR) 3231 s, 2918 s, 2850 s, 1738 s, 1467 w, 1378 w, 1203 ms, 1067 s cm⁻¹; ESI-MS, m/Z (M-2NH₄)²⁻ 619.9, (M-2NH₄—RCOO)⁻ 1011.9, (M-2NH₄+H)⁻ 1240.2.

Method 2. A sample of2-O-benzyl-1,3-bis(1,2-O-dimyristoyl-sn-glycero-3-phosphoryl)glycerol diammonium salt (124.7 mg, 0.09 mmol) was dissolved in THF (15 mL) and hydrogenated with 10% Pd—C (50 mg) overnight under a pressure of 50 psi. After filtration to remove the catalyst, the solution was evaporated to dryness. The residue was dissolved in THF (2 mL), then precipitated using acetone (10 mL). The mixture was kept in freezer overnight and the white solid was filtered and washed with a small amount of cold acetone. After drying in a vacuum desiccator under drierite for 3 h, 98.6 mg of 1,3-bis(1,2-O-dimyristoyl-sn-glycero-3-phosphoryl)glycerol diammonium salt (tetramyristoyl cardiolipin) was obtained. The yield is 85%. TLC (CHCl₃/MeOH/NH₄OH 65:25:5) R_{f}=0.29.

### EXAMPLE 2

### 2A. Synthesis of cis-2-Phenyl-1,3-dioxan-5-yl t-butyldimethylsilyl Ether

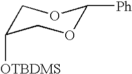

The title compound is prepared from cis-2-phenyl-1,3-dioxan-5-ol according to the procedure described by Dodd et al., J. Chem. Soc. Perkin I, 2273-2277 (1976) with modification. The following is the modified procedure.

To a solution of cis-2-phenyl-1,3-dioxan-5-ol (5.01 g, 27.8 mmol) and imidazole (3.78 g, 55.5 mmol) in DMF (15 mL) was added dimethyl-t-butylsilyl chloride (5.03 g, 33.4 mmol) in portions. The reaction mixture was stirred at rt overnight, then H₂O (20 mL) was added. The mixture was extracted with hexane (25 mL×3). The organic phases were combined, washed with brine, dried over Na₂SO₄ and concentrated in vacuo to give quantitative yield (8.18 g) of cis-2-phenyl-1,3-dioxan-5-yl t-butyldimethylsilyl ether as colorless oil. This product was used in the next step synthesis without further purification.

### 2B. Synthesis of 2-O-t-butyldimethylsilylglycerol

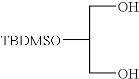

The title compound is prepared from cis-2-phenyl-1,3-dioxan-5-yl t-butyldimethylsilyl ether according to the procedure described by Dodd et al., J. Chem. Soc. Perkin I, 2273-2277 (1976). ¹HNMR (500 MHz, CDCl₃) δ 3.65 (m, 5H, CH₂CHCH₂), 1.88 (t, J=6.0, 2H, OH), 0.92 (s, 9H, SiCCH₃), 0.12 (s, 6H, SiCH₃).

### 2C. Synthesis of Fully Protected Cardiolipin

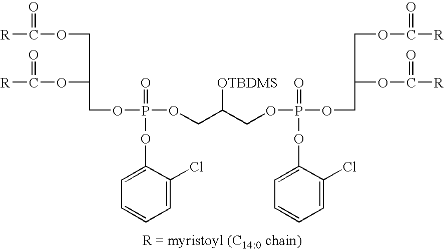

To a solution of o-chlorophenyl dichlorophosphate (1.51 g, 6.15 mmol) and dry pyridine (2.7 mL, 33.3 mmol) in CH₂Cl₂ (6 mL) was added dropwise a solution of 1,2-O-dimyristoyl-sn-glycerol (3.08 g, 6.0 mmol) in CH₂Cl₂ (30 mL) at 0° C. over 15 min. After the reaction mixture was stirred at 0° C. for 1 h and at rt for 1 h, a solution of 2-O-t-butyldimethylsilylglycerol (495.3 mg, 2.4 mmol) in CH₂Cl₂ (6 mL) was added dropwise. The reaction mixture was stirred at rt for 3 h. The organic solvent was removed in vacuo and the remaining residue was treated carefully with cold ethyl acetate (120 mL)/0.25N HCl (120 mL). The organic phase was washed with water, brine, dried over anhydrous Na₂SO₄ and concentrated in vacuo. The obtained residue was purified by flash chromatography on silica gel using hexane/ethyl acetate (4:1 to 3.5:1) to afford 2.79 g of fully protected cardiolipin as a colorless oil. The yield of is 74%. TLC (Hexane/EtOAc 3:1) R_{f}=0.42; ¹HNMR (500 MHz, CDCl₃) δ 7.41 (d, J=8.0 Hz, 4H, ArH), 7.23 (t, J=8.0 Hz, 2H, ArH), 7.12 (t, J=8.0 Hz, 2H, ArH), 5.23 (m, 2H, RCOOCH), 4.36-4.06 (m, 13H, RCOOCH₂, POCH₂, SiOCH), 2.26 (m, 8H, —CH₂COO—), 1.57 (m, 8H, —CH₂CH₂COO—), 1.25 (br s, 80H, CH₂), 0.88 (t, J=6.5, 12H, CH₃), 0.87 (s, 9H, SiCCH₃), 0.08 (s, 6H, SiCH₃).

### 2D. Synthesis of 1,3-Bis(1,2-O-dimyristoyl-sn-glycero-3-phosphoryl)-2-O-(t-butyl dimethylsilyl)glycerol Diammonium Salt

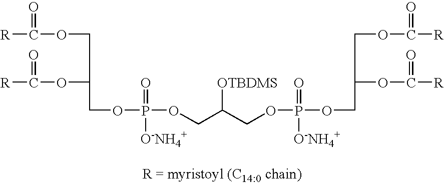

To a stirred solution of fully protected cardiolipin (0.68 g, 0.43 mmol) in THF (10 mL) was added 2-nitrobenzaldoxime (0.71 g, 4.26 mmol) and tetramethylguanidine (0.45 g, 3.91 mmol). After addition of 3 drops of water, the mixture was stirred at rt for 3 h. Solvent was removed in vacuo. The residue was dissolved in CHCl₃ (25 mL) and washed with H₂O (10 mL). The organic phase was dried over Na₂SO₄ and concentrated in vacuo. The obtained yellow residue was purified by flash chromatography on silica gel using CHCl₃/MeOH/NH₄OH (65:15:1) to afford 370 mg of 1,3-bis(1,2-O-dimyristoyl-sn-glycero-3-phosphoryl)-2-O-(t-butyldimethylsilyl)glycerol diammonium salt as a white solid. The yield is 62%. TLC (CHCl₃/MeOH/NH₄OH 65:25:5) R_{f}=0.64; ¹HNMR (500 MHz, CDCl₃) δ 7.32 (br s, NH₄), 5.26 (m, 2H, RCOOCH), 4.34-3.92 (m, 13H, RCOOCH₂, POCH₂, SiOCH), 2.30 (m, 8H, —CH₂COO—), 1.58 (m, 8H, —CH₂CH₂COO—), 1.30 (br s, 80H, CH₂), 0.88 (t, J=6.5, 12H, CH₃), 0.87 (s, 9H, SiCCH₃), 0.08 (s, 6H, SiCH₃).

### 2E. Synthesis of 13 -Bis(1,2-O-dimyristoyl-sn-glycero-3-phosphoryl)glycerol Diammonium Salt (Tetramyristoyl Cardiolipin)

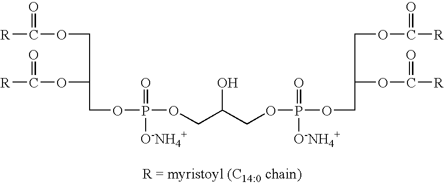

To a stirred mixture of 1,3-bis(1,2-O-dimyristoyl-sn-glycero-3-phosphoryl)-2-O-(t-butyl dimethylsilyl)glycerol diammonium salt (138.0 mg, 0.099 mmol) in CHCl₃ (10 mL), MeOH (20 mL) and H₂O (7 mL) was added dropwise 1N HCl (0.3 mL). The mixture was stirred at rt for 6 h and then, cooled in ice-bath. To the cold reaction mixture, 10% NH₄OH (2 mL) was added dropwise. The organic solvents were removed in vacuo and the remaining aqueous layer was extracted with CHCl₃ twice. The combined organic layer was dried over Na₂SO₄ and concentrated to dryness. The residue was dissolved in THF (5 mL), then precipitated using acetone (25 mL). The mixture was kept in freezer overnight and the white solid was filtered and washed with a small amount of cold acetone. After drying in a vacuum desiccator under drierite for 1 h and under P₂O₅ for 5 h, 115 mg of 1,3-bis(1,2-O-dimyristoyl-sn-glycero-3-phosphoryl)glycerol diammonium salt (tetramyristoyl cardiolipin) was obtained. The yield was 83%. TLC (CHCl₃/MeOH/NH₄OH 65:25:5) R_{f}=0.29. The characterization of tetramyristoyl cardiolipin prepared from Example 2E is identical to that from Example 1C.

### EXAMPLE 3

### 3A. Synthesis of Fully Protected Unsaturated Cardiolipin

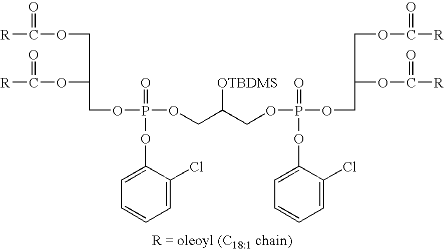

The title compound was prepared according to the method described in Example 2C, substituting 1,2-O-dioleoyl-sn-glycerol in place of 1,2-dimyristoyl-sn-glycerol. The product was a colorless oil with the yield of 35%. TLC (Hexane/EtOAc 3:1) R_{f}=0.46; ¹HNMR (300 MHz, CDCl₃) δ 7.41 (d, J=8.0 Hz, 4H, ArH), 7.23 (t, J=8.0 Hz, 2H, ArH), 7.12 (t, J=8.0 Hz, 2H, Ar), 5.36 (m, 8H, olefinic protons), 5.24 (m, 2H, RCOOCH), 4.35-4.06 (m, 13H, RCOOCH₂, POCH₂, SiOCH), 2.28 (m, 8H, —CH₂COO—), 2.00 (m, 16H, allylic CH₂), 1.57 (m, 8H, —CH₂CH₂COO—), 1.28 (br s, 88H, CH₂), 0.88 (t, J=6.5, 12H, CH₃), 0.88 (s, 9H, SiCCH₃), 0.08 (s, 6H, SiCH₃). ESI-MS, m/z (M+Na)⁺ 1816.4.

### 3B. Synthesis of 1,3-Bis(1,2-O-dioleoyl-sn-glycero-3-phosphoryl)-2-O-(t-butyl dimethylsilyl)glycerol Diammonium Salt

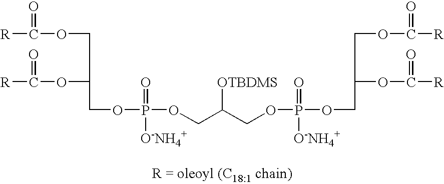

Method 1. To a stirred solution of fully protected unsaturated cardiolipin (170.0 mg, 0.095 mmol), prepared according to the method described in Example 3A, in THF (3 mL) was added 2-pyridinealdoxime (92.7 mg, 0.76 mmol) and tetramethylguanidine (80.6 mg, 0.70 mmol). After addition of 1 drop of water, the mixture was stirred at rt for 7 h. Solvent was removed in vacuo. The residue was dissolved in CHCl₃ (10 mL) and washed with H₂O (4 mL×2), dried over Na₂SO₄ and concentrated in vacuo. The obtained residue was purified by flash chromatography using CHCl₃/MeOH/NH₄OH (65:15:1) to afford 134 mg of 1,3-bis(1,2-O-dioleoyl-sn-glycero-3-phosphoryl)-2-O-(t-butyldimethylsilyl)glycerol diammonium salt as a white gummy solid. The yield of is 88%. TLC (CHCl₃/MeOH/NH₄OH 65:25:5) R_{f}=0.67; ¹HNMR (300 MHz, CDCl₃) δ 7.39 (br s, NH₄), 5.34 (m, 8H, olefinic protons), 5.25 (m, 2H, RCOOCH), 4.31-3.90 (m, 13H, RCOOCH₂, POCH₂, SiOCH), 2.30 (m, 8H, —CH₂COO—), 2.01 (m, 16H, allylic CH₂), 1.59 (m, 8H, —CH₂CH₂COO—), 1.29 (br s, 88H, CH₂), 0.88 (t, J=6.5, 12H, CH₃), 0.87 (s, 9H, SiCCH₃), 0.08 (s, 6H, SiCH₃). ESI-MS, m/z (M-2NH₄)²⁻ 784.8, (M-2NH₄—RCOO)⁻ 1288.3, (M-2NH₄+H)⁻ 1571.9.

Method 2. To a stirred solution of fully protected unsaturated cardiolipin (0.59 g, 0.33 mmol), prepared from the method described in Example 3A, in THF (8 mL) was added 2-nitrobenzaldoxime (0.54 g, 3.23 mmol) and tetramethylguanidine (0.35 g, 3.00 mmol). After addition of 3 drops of water, the mixture was stirred at rt for 3 h. Solvent was removed in vacuo. The residue was dissolved in CHCl₃ (15 mL) and washed with H₂O (6 mL). The organic phase was concentrated in vacuo. The obtained yellow residue was purified by flash chromatography using CHCl₃/MeOH/NH₄OH (65:15:1) to afford 350 mg of 1,3-bis(1,2-O-dioleoyl-sn-glycero-3-phosphoryl)-2-O-(t-butyldimethylsilyl)glycerol diammonium salt as a white gummy solid. The yield is 66%. TLC (CHCl₃/MeOH/NH₄OH 65:25:5) R_{f}=0.67.

### 3C. Synthesis of 1,3-Bis(1,2-O-dioleoyl-sn-glycero-3-phosphoryl)glycerol Diammonium Salt (Tetraoleoyl Cardiolipin)

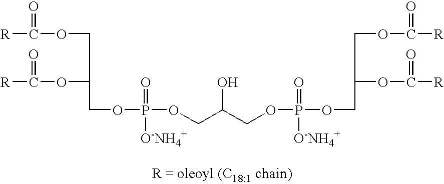

To a stirred mixture of 1,3-bis(1,2-O-dioleoyl-sn-glycero-3-phosphoryl)-2-O-(t-butyl dimethylsilyl)glycerol diammonium salt (110.1 mg, 0.069 mmol) in CHCl₃ (10 mL), MeOH (20 mL) and H₂O (7 mL) was added dropwise 1N HCl (0.3 mL). The mixture was stirred at rt for 5 h. Additional 0.1 mL of 1N HCl was added. The mixture was stirred at rt for additional 4 h and then, cooled in ice-bath. To the cold reaction mixture, 10% NH₄OH (2 mL) was added dropwise. The organic solvents were removed in vacuo and the remaining residue was extracted with CHCl₃. The organic layer was concentrated to dryness. The crude product was purified by flash chromatography on silica gel using CHCl₃/MeOH/NH₄OH (65:15:1) to afford 71 mg of 1,3-bis(1,2-O-dioleoyl-sn-glycero-3-phosphoryl)glycerol diammonium salt (tetraoleoyl cardiolipin) as a white gummy solid. The yield is 70%. TLC (CHCl₃/MeOH/NH₄OH 65:25:5) R_{f}=0.40; ¹HNMR (300 MHz, CDCl₃) δ 7.43 (br s, NH₄), 5.34 (m, 8H, olefinic protons), 5.19 (m, 2H, RCOOCH), 4.38-3.91 (m, 13H, RCOOCH₂, POCH₂, HOCH), 2.29 (m, 8H, —CH₂COO—), 2.17 (br s, 1H, OH), 2.01 (m, 16H, allylic CH₂), 1.58 (m, 8H, —CH₂CH₂COO—), 1.29 (br s, 88H, CH₂), 0.87(t, J=6.5, 12H, CH₃). ESI-MS, m/z (M-2NH₄)²⁻ 727.6, (M-2NH4—RCOO)⁻ 1174.2, (M-2NH₄+H)⁻ 1456.6.

### EXAMPLE 4

### 4A. Synthesis of Fully Protected Cardiolipin

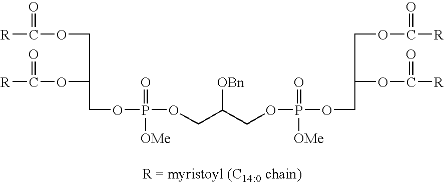

To a solution of N,N-diisopropylmethylphosphonamidic chloride (1.92 g, 9.22 mmol) and dry N,N-diisopropylethylamine (1.92 mL, 11.1 mmol) in CH₂Cl₂ (10 mL) was added dropwise a solution of 1,2-O-dimyristoyl-sn-glycerol (4.61 g, 9.0 mmol) in CH₂Cl₂ (45 mL) at rt over 30 min. After the reaction mixture was stirred at rt for 1.5 h, 1H-tetrazole of 3 wt % solution in acetonitrile (71.8 mL, 24.3 mmol) was added. To this reaction mixture, a solution of 2-benzyloxy-1,3-propanediol (0.66 g, 3.60 mmol) in CH₂Cl₂ (10 mL) was added dropwise. The reaction mixture was stirred at rt for 3 h. The reaction mixture was then cooled to −40° C. and a solution of 77% m-chloroperoxybenzoic acid (2.64 g, 11.80 mmol) in CH₂Cl₂ (10 mL) was added such that the temperature of the reaction mixture was kept below 0° C. On warming to 25° C., the mixture was transferred to a separating funnel and washed with 5% NaHCO₃ (2×50 mL), cold 1N HCl (2×15 mL), water, brine. The organic phase was dried over Na₂SO₄ and concentrated in vacuo to yield an oil residue. The residue was purified by flash chromatography on silica gel eluting with hexane/ethyl acetate (2:1 to 1:1) to afford 4.38 g of fully protected cardiolipin as a colorless oil. The yield is 90%. TLC (Hexane/EtOAc 1:1) R_{f}=0.16; ¹HNMR (300 MHz, CDCl₃) δ 7.35 (m, 5H, ArH), 5.22 (m, 2H, RCOOCH), 4.67 (m, 2H, CH₂Ph), 4.34-4.06 (m, 12H, RCOOCH₂, POCH₂), 3.83 (m, 1H, BnOCH), 3.75 (dt, J₁=11.4, J₂=3.0, 6H, POCH₃), 2.31 (m, 8H, —CH₂COO—), 1.59 (m, 8H, —CH₂CH₂COO—), 1.25 (br s, 80H, CH₂), 0.88 (t, J=6.6, 12H, CH₃).

### 4B. Synthesis of 2-O-Benzyl-1,3-bis(1,2-O-dimyristoyl-sn-glycero-3-phosphoryl)glycerol Diammonium Salt

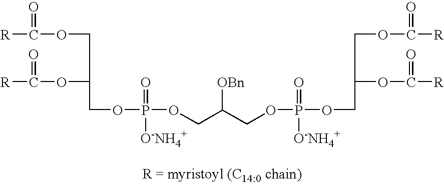

To a stirred solution of fully protected cardiolipin (1.80 g, 1.32 mmol in 2-butanone (85 mL) was added NaI (0.59 g, 3.96 mmol), and the reaction mixture was refluxed for 1.5 h and cooled to 25° C. The resulting white precipitate was filtered and washed with cold 2-butanone to yield 1.71 g of 2-O-benzyl-1,3-bis(1,2-O-dimyristoyl-sn-glycero-3-phosphoryl)glycerol disodium salt as a white solid.

The disodium salt was converted to its corresponding free acid by application of an extraction procedure according to Bligh and Dyer, Can. J. Biochem., 37: 911-917 (1959). Thus, the above disodium salt was dissolved in a cold mixture of CHCl₃ (80 mL), MeOH (160 mL) and 0.1N HCl (80 mL) and stirred at rt for 40 min. Then H₂O (80 mL) and CHCl₃ (80 mL) were added, the separated CHCl₃ layer was isolated and washed with H₂O (50 mL). The organic layer was neutralized by addition of 15 mL of 10% NH₄OH. The organic layer was separated and concentrated in vacuo to give residue, which was further purified through a short silica gel column using CHCl₃/MeOH/NH₄OH (65:15:1) to afford 1.42 g of 2-O-benzyl-1,3-bis(1,2-O-dimyristoyl-sn-glycero-3-phosphoryl)glycerol diammonium salt as a white solid. The yield was 79%. TLC (CHCl₃/MeOH/NH₄OH 65:25:5) R_{f}=0.64. The characterization of final product prepared from Example 4B is identical to that from Example 1B.

### 4C. Synthesis of 1,3-Bis(1,2-O-dimyristoyl-sn-glycero-3-phosphoryl)glycerol Diammonium Salt (Tetramyristoyl Cardiolipin)

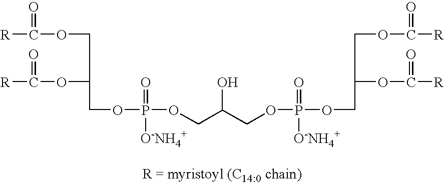 The title compound is prepared according to the method described in Example 1C. The characterization of tetramyristoyl cardiolipin prepared from the method described in Example 4 is identical to that from Example 1.

### EXAMPLE 5

### 5A. Synthesis of 2-O-Benzyl-13-bis(1,2-O-dimyristyl-sn-glycero-3-phosphoryl)glycerol Dimethyl Ester

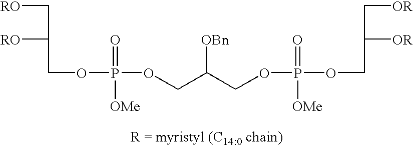

To a stirred solution of N,N-diisopropylmethylphosphonamidic chloride (1.02 g, 5.15 mmol) and dry N,N-diisopropylethylamine (1.2 mL, 6.94 mmol) in CH₂Cl₂ (4 mL) was added dropwise a solution of 1,2-O-dimyristyl-sn-glycerol (2.00 g, 4.13 mmol) in CH₂Cl₂ (20 mL) at rt over 15 min. After the reaction mixture was stirred at rt for 1.5 h, 1H-tetrazole of 3 wt % solution in acetonitrile (31.0 mL, 10.5 mmol) was added. To this reaction mixture, a solution of 2-benzyloxy-1,3-propanediol (0.30 g, 1.65 mmol) in CH₂Cl₂ (5 mL) was added. The reaction mixture was stirred at rt for 3 h. The reaction mixture was then cooled to −40° C. and a solution of 77% m-chloroperoxybenzoic acid (1.14 g, 6.6 mmol) in CH₂Cl₂ (7 mL) was added. The mixture was gradually warmed to rt for 30 min, then transferred to a separating funnel and washed with 5% NaHCO₃ (2×30 mL), 1N HCl (2×20 mL), water, brine. The organic phase was dried over Na₂SO₄ and concentrated in vacuo. The residue was purified by flash chromatography on silica gel eluting with a gradient of hexane/ethyl acetate (1:0 to 1:1) to afford 1.69 g of 2-O-benzyl-1,3-bis(1,2-O-dimyristyl-sn-glycero-3-phosphoryl)glycerol dimethyl ester. The yield is 79%. TLC (Hexane/EtOAc 1:1) R_{f}=0.24. ¹HNMR (300 MHz, CDCl₃) δ 7.35-7.29 (m, 5H, ArH), 4.68 (m, 2H, CH₂Ph), 4.26-4.02 (m, 8H, POCH₂), 3.86 (m, 2H, ROCH), 3.75 (d, J₁=12.0, 6H, POCH₃), 3.61-3.38 (m, 13H, —CH₂OCH₂—, —CH₂OCH—, BnOCH), 1.54 (m, 8H, —CH₂CH₂O—), 1.29 (m, 88H, CH₂), 0.88 (t, J=6.7, 12H, CH₃).

### 5B. Synthesis of 2-O-Benzyl-1,3-bis(1,2-O-dimyristyl-sn-glycero-3-phosphoryl)glycerol Diammonium Salt

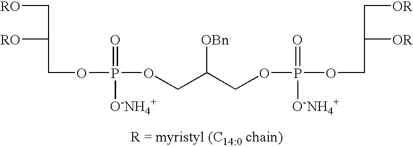

To a stirred solution of 2-O-benzyl-1,3-bis(1,2-O-dimyristyl-sn-glycero-3-phosphoryl)glycerol dimethyl ester (230 mg, 0.18 mmol) in 2-butanone (4 mL) was added NaI (88 mg, 0.59 mmol). The reaction mixture was refluxed for 1.5 h and cooled to 25° C. and then at 0° C. The resulting solid precipitate was filtered and washed with cold 2-butanone to yield 2-O-benzyl-1,3-bis(1,2-O-dimyristyl-sn-glycero-3-phosphoryl)glycerol disodium salt. The disodium salt was taken in chloroform/methanol/0.1N HCl (30:5:15 mL) and vigorously stirred for 1 h. The organic layer was separated and the aqueous layer was extracted with chloroform (2×10 mL). The combined organic extracts were washed with water (2×10 mL). Aqueous ammonium hydroxide (5 mL) was added to the chloroform extract and concentrated in vacuo and dried overnight under high vacuum to afford 231 mg of 2-O-benzyl-1,3-bis(1,2-O-dimyristyl-sn-glycero-3-phosphoryl)glycerol diammonium salt. The yield is 60%. TLC (CHCl₃/MeOH/NH₄OH, 65:25:5) R_{f}=0.5; ¹H NMR (CDCl₃): δ 7.29-7.21 (m, 5H, ArH), 4.57 (m, 2H, CH₂Ph), 4.21-3.38 (m, 23H, POCH₂, —CH₂OCH₂—; —CH₂OCH—, BnOCH), 1.50 (m, 8H, CH₂CH₂O—), 1.25 (m, 88H, CH₂), 0.89 (t, 12H, J=6.54 Hz, CH₃); ESI-MS, m/z (M-2NH₄+H)⁻, 1274.1, (M-2NH₄)²⁻ 636.9.

### 5C. Synthesis of 1,3-bis(1,2-O-dimyristyl-sn-glycero-3-phosphoryl)glycerol Diammonium Salt. (Tetramyristyl Cardiolipin Ether Analogue)

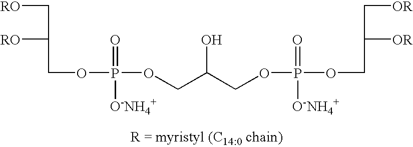

2-O-Benzyl-1,3-bis(1,2-O-dimyristyl-sn-glycero-3-phosphoryl)glycerol diammonium salt (85 mg, 0.065 mmol) was dissolved in tetrahydrofuran (10 mL). To this, 10% Pd—C (70 mg) was added and the mixture was shaken on Parr hydrogenator at 50 psi for 16 hrs. The solution was filtered through a pad of celite and washed with chloroform (5 mL). The filtrate and the washings were combined, concentrated in vacuo and dried. After crystallization from tetrahydrofuran (0.2 mL)-acetone (8 mL) mixture, 44 mg of 1,3-bis(1,2-O-dimyristyl-sn-glycero-3-phosphoryl)glycerol diammonium salt. The yield is 56%. TLC (CHCl₃/MeOH/NH₄OH, 65:25:5) R_{f}=0.28; ¹H NMR (CDCl₃): δ 7.29 (br, NH₄⁺), 4.20-3.80 (m, 8H, POCH₂), 3.57-3.41 (m, 15H, CH₂OCH₂; CH₂OCH—, HOCH), 2.3 (br, 1H, OH), 1.53 (m, 8H, CH₂CH₂O—), 1.25 (m, 88H, CH₂), 0.875 ((t, 12H, J=6.9 Hz, CH₃); ESI-MS, m/z 1184.7 (M-2NH₄+H)⁻, 591.3 (M-2NH₄)²⁻.

### EXAMPLE 6

### 6A. Synthesis of 2-O-Benzyl-1,3-bis(1,2-O-dimyristoyl-sn-glycero-3-phosphoryl)glycerol Diammonium Salt

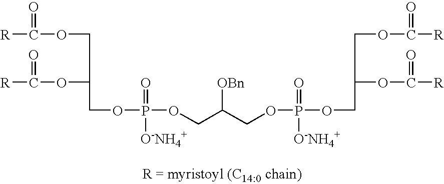

To a stirred solution of N,N′-dicyclohexylcarbodimide (265 mg, 1.28 mmol) in pyridine (2 mL) was added dropwise 1,2-O-dimyristoyl-sn-glycero-3-phosphatidic acid (281 mg, 0.47 mmol) in anhydrous CH₂Cl₂ (3 mL). The solution was stirred for 5 min at rt, and then 2-benzyloxy-1,3-propanediol (0.71 g, 3.90 mmol) in CH₂Cl₂ (8 mL) was added and the stirring continued at rt for 24 h. The reaction was filtered and washed with CH₂Cl₂. The filtrate was concentrated and co-evaporated with toluene to remove traces of pyridine. To the residue, CH₂Cl₂ (15 mL) was added and the mixture was stored in the freezer overnight. The white precipitation was removed by filtration. The filtrate was concentrated in vacuo and purified on silica gel column eluting with CHCl₃/MeOH/NH₄OH (65:15:1). Yield 13 mg (4%). TLC (CHCl₃/MeOH/NH₄OH 65:25:5) R_{f}=0.64. The TLC was identical with the authentic sample prepared according to the method described in Example 1B.

### 6B. Synthesis of 1,3-Bis(1,2-O-dimyristoyl-sn-glycero-3-phosphoryl)glycerol Diammonium Salt (Tetramyristoyl Cardiolipin)

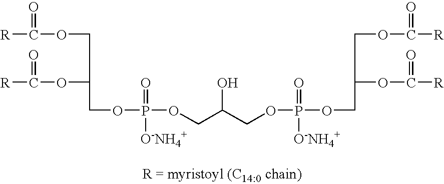

The title compound is prepared according to the method described in Example 1C. TLC (CHCl₃/MeOH/NH₄OH 65:25:5) R_{f}=0.29. The TLC was identical with the authentic sample prepared according to the method described in Example 1C.

### EXAMPLE 7

This example demonstrates preparation of a cardiolipin-containing liposome composition of the invention. Small unilamellar vesicles are formed by mixing 19.1 μmole tetramyristoyl cardiolipin, as prepared above, 96.2 μmol phosphatidyl choline and 64.6 μmol cholesterol. After thorough stirring, the mixture is evaporated to dryness in a 50 ml round-bottom flask using a rotary evaporator. The subsequent dried lipid film is resuspended in 10 ml sterile non-pyrogenic water. After a 30 min swelling time, the resulting suspension is sonicated in a fixed temperature bath at 25° C. for 15 min. The preparation of liposomes is then lyophilized with trehalose.

### EXAMPLE 8

This example demonstrates the preparation of liposomes including the tetramyristoyl cardiolipin, as prepared above that retain the anthracycline, doxorubicin. Liposomal doxorubicin can be prepared for clinical administration by simple vortex mixing of a vial containing 40 mg cardiolipin-liposome lyophilizate and 2.5 ml of a doxorubicin solution previously prepared in 0.85% NaCl at 2 mg/ml. Vortex mixing is completed for 1 minute and mixture is kept at 37° C. for a 15 min period incubation.

### EXAMPLE 9

This example demonstrates the preparation of liposomes that retain the drug mitoxantrone HCl. A lipid mixture is prepared by mixing 1.96 gm D-α-tocopherol, 58.7 gm tetramyristoyl cardiolipin, as prepared above, 97.9 gm cholesterol, 293.6 gm egg phosphatidylcholine in t-butyl alcohol so that the solution weighs a total of 13.05 kg. A 3,080 gm aqueous solution containing 122.4 gm of trehalose dihydrate is then mixed into the butyl alcohol solution. Vials are filled with 11.1 gm of this mixture and lyophilized such that about 300 mg of lipid is contained in each vial. 7.5 ml of Novantroneg (15 mg) and 7.5 ml of water are added to the lipid vials to prepare the liposome encapsulated mitoxantrone. The liposomes are allowed to hydrate at room temperature for 30 minutes, vortexed vigorously for 2 min, and sonicated for 10 min at maximum intensity. A suitable quantity is dispensed in a syringe or standard infusion set over a period of 45 min for use within 8 hours.

### EXAMPLE 10

This example demonstrates the preparation of liposomes that retain the drug paclitaxel. Paclitaxel can be encapsulated in liposomes of cardiolipin, phosphatidylcholine, cholesterol and α-tocopherol. The proportion of lipids per mg of paclitaxel is:
1.8 mg cardiolipin
9.0 mg phophatidylcholine
3.0 mg cholesterol
0.1 mg α-tocopheryl

The liposome encapsulated paclitaxel can be manufactured by adding 8.89 kilograms of t-butyl alcohol to a 12.0 liter flask and heating it to 40-45° C. The following additions are made sequentially with mixing until dissolution and heating at 40-45° C.: 3.412 grams of D-α-tocopheryl acid succinate, 205 grams of egg phosphatidylcholine, 22.78 grams of paclitaxel, 41.00 grams of tetramyristoyl cardiolipin as prepared above, 68.33 grams of cholesterol.

The resulting solution is filtered through a 0.22 micron filter. The resulting filtrate is filled into sterile vials, each containing about 10.1 grams of filtrate. The vials are stoppered and subjected to lyophilization. They can be stored at −20° C. until use.

Liposomes are prepared from the dry lipid film, as needed, with 25 ml of normal saline solution. The mixture is allowed to hydrate at room temperature for about one hour, after which time the vials are vortexed for about one minute and sonicated for about 10 minutes in a bath type sonicator at maximum frequency. An appropriate amount of the contents of the vial can be transferred to an infusion bag and infused into a patient in accordance with the present invention.

This liposomal formulation of paclitaxel can be used to rapidly administer a large quantity of taxane to humans without inducing a substantial toxic reaction. Treatments can be administered intravenously over a period of about an hour, or even a 45 min, or less. At least three patients were treated at about the following dosages: 90 mg/m², 135 mg/m², 175 mg/m², 250 mg/m², and 300 mg/m², allowing for normal laboratory and therapeutic dose variation. The formulation can be given as a single agent without pretreatment with steroids, antihistamines or other therapeutic agents such as anaphylaxis inhibitors. Treatments can be repeated every 21 days as patient tolerance permits.

### EXAMPLE 11

This example demonstrates the preparation of liposomes that contain SN-38 in solution. A lipid film is prepared by adding about 0.2 g of D-α-tocopherol acid succinate to about 1 kg of t-butyl alcohol which is warmed to about 35-40° C. The solution is mixed for about 5 min until the tocopherol is dissolved. About 6.0 g of tetramyristoyl cardiolipin, as prepared above, is added to the solution and the solution is mixed for about 5 minutes. About 10 g of cholesterol is added to the solution and the solution is mixed for about 5 more minutes then about 30 g of egg phosphatidyl choline is added followed by mixing for another 5 min. Approximately 11 grams of the resulting lipid solution is lyophilized to generate a lipid film.

To prepare liposomal SN-38, a 1.2 mg/ml solution of SN-38 is prepared by dissolving the drug in an aqueous alkaline solution having a pH of between 8 and 10. Approximately 15 ml of this SN-38 solution is added to a vial containing the lipid film. The vial is swirled gently, allowed to hydrate at room temperature for 30 min, vortexed vigorously for 2 min, and sonicated for 10 min in a bath-type sonicator at maximum intensity. The pH of the liposome solution is reduced to acid pH. Using this method more than 90 wt. % of the SN-38 is complexed with lipid in the form of liposomes.

### REFERENCES CITED

1. Bruzik, K. S.; Kubiak, R. J. Tetrahedron Lett., 36: 2415-2418 (1995).
2. DeHaas, G. H.; Bonsen, P. P. M.; VanDeenen, L. L. M. Biochim. Biophys. Acta, 116: 114-124 (1966).
3. Dodd, G. H.; Dolding, B. T.; Ioannou, P. V. J. Chem. Soc. Perkin I, 21: 2273-2277 (1976).
4. Duralski, A. A.; Spooner, P. J. R.; Rankin, S. E.; Watts, A. Tetrahedron Lett., 39: 1607-1610 (1998).
5. Inoue, K.; Nojima, S. Chem. Pharm. Bull., 16: 76-81 (1968).
6. Inoue, K.; Suhara, Y.; Nojima, S. Chem. Pharm. Bull., 11: 1150-1156 (1963).
7. Ioannou, P. V.; Golding, B. T. Prog. Lipid Res. 17: 279-318 (1979).
8. Ioannou, P. V.; Marecek, J. F. Chem. Chron. 15: 205-220 (1986).
9. Keana, J. F.; Shimizu, M.; Jemstedt, K. K. J. Org. Chem. 51: 2297-2299 (1986).
10. Mishina, I. M.; Vasilenko, I. A.; Stepanov, A. E.; Shvets, V. I. Bioorg. Khim., 11: 992-994 (1985).
11. Mishina, I. M.; Vasilenko, I. A.; Stepanov, A. E.; Shvets, V. I. Bioorg. Khim., 13: 1110-1115 (1987).
12. Moschidis, M. C. Chem. Phys. Lipids, 46: 253-257 (1988).
13. Ramirez, F.; Ioannou, P. V.; Marecek, J. F.; Golding, B. T.; Dodd, G. H. Synthesis, 769-770 (1976).
14. Ramirez, F.; Ioannou, P. V.; Marecek, J. F.; Dodd, G. H.; Golding, B. T. Tetrahedron, 33: 599-608 (1977).
15. Saunders, R. M.; Schwarz, H. P. J. Am. Chem. Soc. 88: 3844-3847 (1966). Stepanov, A. E.; Makarova, I. M.; Shvets, V. I. Zh. Org. Khim., 20: 985-988 (1984).

All references, including publications, patent applications, and patents, cited herein, including those in the preceding list and otherwise cited in this Specification, are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments can become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1.-70. (canceled)

71. A method for preparing a cardiolipin or cardiolipin analogue, comprising reacting phosphatidic acid and 2-O-protected glycerol in the presence of a coupling agent, which is N,N′-dicyclohexylcarbodimide or N,N′-carbonyldimidazole.

72. A method for preparing a cardiolipin or cardiolipin analogue, comprising reacting phosphatidic acid and glycerol in the presence of a coupling agent, which is triisopropylbenzenesulfonyl chloride, N,N′-dicyclohexylcarbodiimide or N,N′-carbonyldimidazole.

73. A method for preparing a cardiolipin or cardiolipin analogue comprising reacting an alcohol of the formula V and 2-O-protected glycerol or 2-O-substituted glycerol in the presence of a coupling agent, which is either dichlorophosphate or N,N-diisopropylmethylphosphonamidic chloride. 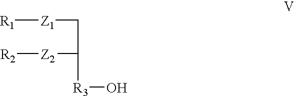
wherein Z₁ and Z₂ are the same or different and are —O—C(O)—, —O—, —S—, —NH—C(O)—R₁ and R₂ are the same or different and are H and/or a saturated or unsaturated alkyl group;
R₃ is (CH₂)ₙ and n=0-10.

74. The method of any of claims 71-73, wherein the cardiolipin or cardiolipin analogue has the structure of I, II, III or IV: 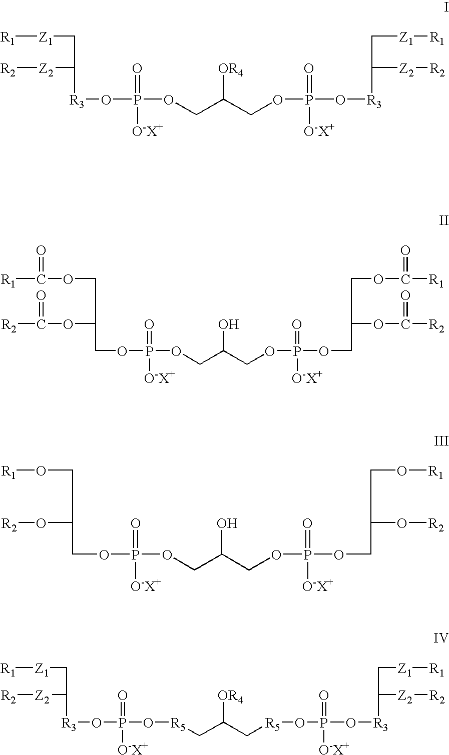
wherein Z₁ and Z₂ are the same or different and are —O—C(O)—, —O—, —S—, —NH—C(O)—;
R₁ and R₂ are the same or different and are H or a saturated or unsaturated alkyl group;
R₃ is (CH₂)ₙ and n=0-10;
R₄ is hydrogen, alkyl, substituted alkyl, cycloalkyl, substituted cycloalkyl, a peptide, dipeptide, polypeptide, protein, carbohydrate such as glucose, mannose, galactose, polysaccharide and the like, heterocyclic, nucleoside , or a polynucleotide;
R₅ is a linker;
X is a cation.

75. The method of claim 74, wherein the linker comprises alkyl, substituted alkyl, cycloalkyl, substituted cycloalkyl, alkyloxy, polyalkyloxy such as pegylated ether of containing from 1 to 500 alkyloxy mers, substituted polyalkyloxy and the like, a peptide, dipeptide, polypeptide, protein, carbohydrate such as glucose, mannose, galactose, and polysaccharides.

76. The method of claim 74, wherein at least one of R₁ and/or R₂ is a saturated or unsaturated alkyl group having between 4 and 34 carbons.

77. The method of claim 74, wherein at least one of R₁ and/or R₂ is a saturated or unsaturated alkyl group having between 14 and 34 carbons.

78. The method of claim 74, wherein at least one of R₁ and/or R₂ is a saturated or unsaturated alkyl group having between 14 and 24 carbons.

79. The method of claim 74, wherein X is a non-toxic cation.

80. The method of claims 74, wherein X is selected from a group consisting of hydrogen, ammonium, sodium, potassium, calcium, or barium ion.

81. A method for preparing the cardiolipin or cardiolipin analogue of structure II, comprising reacting 1,2-O-diacyl glycerol and 2-O-protected glycerol in the presence of a coupling agent, which is either dichlorophosphate or N,N-diisopropylmethylphosphonamidic chloride.

82. A method for preparing the cardiolipin or cardiolipin analogue of structure III, comprising reacting 1,2-O-dialkyl glycerol and 2-O-protected glycerol in the presence of a coupling agent, which is either dichlorophosphate or N,N-diisopropylmethylphosphonamidic chloride.

83. A method for preparing a cardiolipin or cardiolipin analogue of structure IV, comprising reacting an alcohol of formula V and a diol of the formula VI, wherein R₄ and R₅ are as defined in claim 74, in the presence of a coupling agent, which is either dichlorophosphate or N,N-diisopropylmethylphosphonamidic chloride.

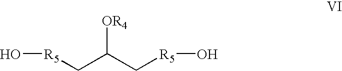

84. The method of any of claims 73, 81, 82 or 83, wherein the dichlorophosphate is of the formula VII: 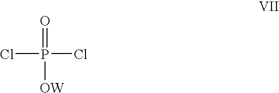
wherein W is alkyl groups or substituted alkyl groups including methyl, ethyl, isopropyl, t-butyl, allyl, 2-substituted ethyl, haloethyl such as 2,2,2-tribromoethyl; benzyl or substituted benzyl groups; phenyl or substituted phenyl groups such as 2-chlorophenyl, 4-chlorophenyl and 2,4-dichlorophenyl; or any other removable protecting groups.

85. A cardiolipin or cardiolipin analogue produced in accordance with the method of any of claims 71-73 or 75-83.

86. The cardiolipin or cardiolipin analogue of claim 85, wherein said cardiolipin or cardiolipin analogue has a chain length of C₄-C₁₃.

87. The cardiolipin or cardiolipin analogue of claim 85, wherein said cardiolipin or cardiolipin analogue has a chain length of C₁₅-C₃₄.

88. A method for preparing a liposome, comprising preparing a cardiolipin or a cardiolipin analogue by any of the methods of claims 71-73 or 75-83 and then including said cardiolipin or cardiolipin analogue in a liposome.

89. A method of retaining an active agent in a liposome, comprising preparing a cardiolipin or cardiolipin analogue by the method of any of claims 71-73 or 75-83 and including said cardiolipin or cardiolipin analogue and an active agent in a liposome.

90. The method of claim 89, wherein the active agent becomes entrapped within the liposomes.

91. The method of claim 89, wherein the active agent becomes complexed with the cardiolipin or cardiolipin analogue.

92. The method of claim 88, further comprising lyophilizing the liposomes.

93. The method of claim 89, further comprising lyophilizing the liposomes.

94. A liposomal composition comprising cardiolipin or cardiolipin analogues prepared in accordance with the method of claim 88.

95. A liposomal composition comprising cardiolipin or cardiolipin analogues prepared in accordance with the method of claim 89.

96. The liposomal preparation of any of claims 94-95, wherein the cardiolipin or cardiolipin analogue comprises a chain length of C₄-C₁₃.

97. The liposomal preparation of any of claims 94-95, wherein the cardiolipin or cardiolipin analogue comprises a chain length of C₁₅-C₃₄.

98. A composition comprising cardiolipin analogues prepared in accordance with the method of any of claims 71-73 or 75-83.

99. The composition of claim 108, wherein the cardiolipin analogue comprises a chain length of C₄-C₁₃.

100. The composition of claim 98, wherein the cardiolipin analogue comprises a chain length of C₁₅-C₃₄.

101. A liposomal composition comprising a cardiolipin or a cardiolipin analogue of claim 85, in liposomal form and an active agent.

102. A use of the composition of claim 85 to prepare a medicament for treatment of a disease.

103. A use of the composition of any of claims 94 or 95 to prepare a medicament for treatment of a disease.

104. A method of delivering an active agent to a cell, comprising preparing a composition according to any of claims 71-73 or 75-83 and exposing the composition to a cell.

105. A method of delivering an active agent to a cell, comprising preparing a composition according to claim 89 and exposing the composition to a cell.

106. A method of treating a human or animal disease, comprising preparing a composition according to any of claims 71-73 or 75-83 and exposing the composition to a human or animal in need thereof such that the active agent is delivered to the human or animal patient.

107. A method of treating a human or animal disease, comprising preparing a composition according to claim 88 and exposing the composition to a human or animal in need thereof such that the active agent is delivered to the human or animal patient.

108. A method of treating a human or animal disease, comprising preparing a composition according to claim 89 and exposing the composition to a human or animal in need thereof such that the active agent is delivered to the human or animal patient.
